(19) United States
(12) Patent Application Publication
(10) Pub. No.: US 2026/0275368 A1
(43) Pub. Date: Sep. 17, 2026

(54) COUMARIN SYNTHESIS AND USES THEREOF

(71) Applicant: BASF SE, Ludwigshafen (DE)

(72) Inventors: Uwe Conrath, Aachen (DE); Caspar Langenbach, Aachen (DE); Sebastian Felix Beyer, Limburgerhof (DE); Patrick Schwinges, Aachen (DE); Holger Schultheiss, Einbeck (DE); Alexander Beesley, Aachen (DE); Cedric Linke, Aachen (DE)

(21) Appl. No.: 18/838,839

(22) PCT Filed: Feb. 8, 2023
(86) PCT No.: PCT/EP2023/053094

§ 371 Date: Aug. 15, 2024

(30) Foreign Application Priority Data
Feb. 18, 2022 (EP) 22157545.9

(51) Int. Cl. C12N 15/82; C12N 5/04; C12N 9/02; C12Q 1/68

(52) U.S. Cl. CPC C12N 15/8243; C12N 5/04; C12N 9/0071; C12N 15/8225; C12N 15/8226; C12N 15/8227; C12N 15/8279; C12Q 1/68; C12Y 114/11; C12Y 114/14; C12N 2510/00; C12N 2830/34; C12N 2830/36; C12Q 2600/13

(57) ABSTRACT

The present invention relates to genes, materials and methods for coumarin synthesis and the application thereof for improving plant health, preferably against infection by phythopathogenic microorganisms, and/or for improving plant health against coumarin-induced adverse effects on plant health. Furthermore, the invention pertains to methods and uses of such genes and materials for creating correspondingly beneficial plant cells, plant parts and whole plants, and relates to products obtained from such plants or plant parts.

The present invention relates to genes, materials and methods for coumarin synthesis and the application thereof for improving plant health, preferably against infection by phythopathogenic microorganisms, and/or for improving plant health against coumarin-induced adverse effects on plant health. Furthermore, the invention pertains to methods and uses of such genes and materials for creating correspondingly beneficial plant cells, plant parts and whole plants, and relates to products obtained from such plants or plant parts.

### BACKGROUND OF THE INVENTION

Plant pathogenic organisms and particularly fungi have resulted in severe reductions in crop yield in the past, in worst cases leading to famine. Monocultures in particular are highly susceptible to an epidemic-like spreading of diseases. To date, the pathogenic organisms have been controlled mainly by using pesticides. Currently the possibility of directly modifying the genetic disposition of a plant or pathogen is also open to man. Alternatively, naturally occurring fungicides produced by the plants after fungal infection can be synthesized and applied to the plants.

The term “resistance” as used herein refers to an absence or reduction of one or more disease symptoms in a plant caused by a plant pathogen. Resistance generally describes the ability of a plant to prevent, or at least curtail the infestation and colonization by a harmful pathogen. Different mechanisms can be discerned in the naturally occurring resistance, with which the plants fend off colonization by phytopathogenic organisms (Schopfer and Brennicke (1999) Pflanzen-physiologie, Springer Verlag, Berlin-Heidelberg, Germany).

With regard to resistance, a differentiation is made between compatible and incompatible interactions. In the compatible interaction, an interaction occurs between a virulent pathogen and a susceptible plant. The pathogen survives and may build up reproductive structures, while the host is seriously hampered in development or dies off. An incompatible interaction occurs, on the other hand, when the pathogen infects the plant but is inhibited in its growth before or after weak development of symptoms (mostly by the presence of Resistance (R) genes of the NBS-LRR family, see below). In the latter case, the plant is resistant to the respective pathogen (Schopfer and Brennicke, vide supra). However, this type of resistance is mostly specific for a certain strain or pathogen.

In both compatible and incompatible interactions, a defensive and specific reaction of the host to the pathogen occurs. In nature, however, this resistance is often overcome because of the rapid evolutionary development of new virulent races of the pathogens (Neu et al. (2003) American Cytopathol. Society, MPMI 16 No. 7:626-633).

Most pathogens are plant species specific. This means that a pathogen can induce a disease in a certain plant species, but not in other plant species (Heath (2002) Can. J. Plant Pathol. 24: 259-264). The resistance against a pathogen in certain plant species is called non-host resistance. The non-host resistance offers strong, broad, and permanent protection from phytopathogens. Genes providing non-host resistance provide the opportunity of a strong, broad and permanent protection against certain diseases in non-host plants. In particular, such a resistance works for different strains of the pathogen.

Fungi are distributed worldwide. Approximately 100 000 different fungal species are known to date. Thereof, rusts are of great importance. They can have a complicated development cycle with up to five different spore stages (spermatium, aecidiospore, uredospore, teleutospore and basidiospore).

During the infection of plants by pathogenic fungi, different phases are usually observed. The first phases of the interaction between phytopathogenic fungi and their potential host plants are decisive for the colonization of the plant by the fungus. During the first stage of the infection, the spores become attached to the surface of the plants, germinate, and the fungus penetrates the plant. Fungi may penetrate the plant via existing ports such as stomata, lenticels, hydathodes and wounds, or else they penetrate the plant epidermis directly as the result of mechanical force with the aid of cell wall digesting enzymes. Specific infection structures are developed for penetration of the plant. To counteract, plants have developed physical barriers, such as wax layers, and chemical compounds having antifungal effects to inhibit spore germination, hyphal growth or penetration.

The soybean rust Phakopsora pachyrhizi directly penetrates the plant epidermis. After growing through the epidermal cell, the fungus reaches the intercellular space of the mesophyll, where the fungus starts to spread through the leaf. To acquire nutrients, the fungus penetrates mesophyll cells and develops haustoria inside the mesophyll cells. During the penetration process the plasma membrane of the penetrated mesophyll cell stays intact. It is a particularly troubling feature of Phakopsora rusts that these pathogens exhibit an immense variability, thereby overcoming novel plant resistance mechanisms and novel fungicide activities within a few years and sometimes already within one Brazilian growing season.

Fusarium species are important plant pathogens that attacks a wide range of plant species including many important crops such as maize and wheat. They cause seed rots and seedling blights as well as root rots, stalk rots and ear rots. Pathogens of the genus Fusarium infect the plants via roots, silks or previously infected seeds or they penetrate the plant via wounds or natural openings and cracks. After a very short establishment phase the Fusarium fungi start to secrete mycotoxins such as trichothecenes, zearalenone and fusaric acid into the infected host tissues leading to cell death and maceration of the infected tissue. Feeding on dead tissue, the fungus then starts to spread through the infected plant leading to severe yield losses and decreases in quality of the harvested grain.

Biotrophic phytopathogenic fungi depend for their nutrition on the metabolism of living plant cells. This type of fungi belongs to the group of biotrophic fungi, like many rust fungi, powdery mildew fungi or oomycete pathogens like the genus Phytophthora or Peronospora. Necrotrophic phytopathogenic fungi depend for their nutrition on dead cells of the plants, e.g. species from the genus Fusarium, Rhizoctonia or Mycosphaerella. Soybean rust occupies an intermediate position. It penetrates the epidermis directly, whereupon the penetrated cell becomes necrotic. However, after penetration, the fungus changes over to an obligate-biotrophic lifestyle. The subgroup of the biotrophic fungal pathogens which follows essentially such an infection strategy are heminecrotrophic.

Coumarins are antimicrobial phenolic compounds that can act as phytoanticipins or phytoalexins in plants. Coumarins are produced, for example, upon infection, injury, heat treatment, gamma and ultraviolet irradiation. They have been associated with basal, gene-for-gene, and induced resistance to insects, fungi and other microbes, viruses and postharvest decay (Stringlis, I. A., De Jonge, R. & Pieterse, C. M. J. The Age of Coumarins in Plant-Microbe Interactions. Plant Cell Physiol. 60, 1405-1419 (2019); Chen, J., Shen, Y., Chen, C. & Wan, C. Inhibition of key citrus postharvest fungal strains by plant extracts in vitro and in vivo: A review. Plants 8, 1-19 (2019); Venugopala, K. N., Rashmi, V. & Odhav, B. Review on natural coumarin lead compounds for their pharmacological activity. Biomed Res. Int. 2013, (2013)). While in most plants coumarins are produced in the cytoplasm and are only secreted upon iron deficiency (in Arabidopsis roots, Fourcroy, P. et al. Involvement of the ABCG37 transporter in secretion of scopoletin and derivatives by Arabidopsis roots in response to iron deficiency. New Phytol. 201, 155-167 (2014)) or disruption of the tissue (in tobacco hypersensitive response; Costet, L., Fritig, B. & Kauffmann, S. Scopoletin expression in elicitor-treated and tobacco mosaic virus-infected tobacco plants. Physiol. Plant. 115, 228-235 (2002)), leaves of the common sunflower (Helianthus annuus L.) can achieve targeted coumarin export to the epidermal surface, inhibiting fungal pathogens such as Alternaria helianthi and Puccinia spec. (Prats, E., Llamas, M. J., Jorrin, J. & Rubiales, D. Constitutive coumarin accumulation on sunflower leaf surface prevents rust germ tube growth and appressorium differentiation. Crop Sci. 47, 1119-1124 (2007)). Sunflower cultivars with increased concentrations of the coumarins scopoletin and ayapin (6,7-[methylenedioxy]coumarin) have enhanced resistance to disease (Tal, B. & Robeson, D. J. The Metabolism of Sunflower Phytoalexins Ayapin and Scopoletin. Plant Physiol. 82, 167-172 (1986)). In addition to fungal inhibition, decreased susceptibility of sunflower varieties to phytopathogenic oomycetes and parasitic plants was also linked to enhanced coumarin accumulation (Leon, A., Jorrin-novo, J. V & Novo, J. Agronomic Aspects of the Sunflower 7-Hydroxylated Simple Coumarins 7-Hydroxylated Simple Coumarins. (2016); Gascuel, Q. et al. The sunflower downy mildew pathogen Plasmopara halstedii. Mol. Plant Pathol. 16, 109-122 (2015); Serghini, K., Pérez De Luque, A., Castejón-Muñoz, M., García-Torres, L. & Jorrín, J. V. Sunflower (Helianthus annuus L.) response to broomrape (Orobanche cernua Loefl.) parasitism: Induced synthesis and excretion of 7-hydroxylated simple coumarins. J. Exp. Bot. 52, 2227-2234 (2001)).

In the past, attempts were made to produce or accumulate a class of antifungal endogenous secondary metabolites called coumarins in plants, particularly scopoletin, scopolin, esculetin, isoscopoletin, and scoparone. Genes, materials and methods for the synthesis of coumarins in plants and their transport to leaf surfaces are described in WO2016124515, WO2020120753 and EP21197814 (application number) which are incorporated herein by reference. While expression of, for example, scopoletin was highly effective at reducing fungal infections, it was also observed that constitutively strong expression of scopoletin biosynthesis genes can lead to reduced overall plant health and yield, particularly in soybean.

In plants, the coumarin scopoletin can be further metabolised to yield modified simple coumarins, all with various functions in the plants defence. When adding a hydroxyl-group at the 8′ C atom of scopoletin, catalysed by the scopoletin 8-hydroxylase (S8H), fraxetin is formed (Siwinska J, Siatkowska K, Olry A, Grosjean J, Hehn A, et al. Scopoletin 8-hydroxylase: A novel enzyme involved in coumarin biosynthesis and iron-deficiency responses in Arabidopsis. J Exp Bot 2018; 69:1735-1748.).

Due to the catecholic properties of fraxetin, it has been linked to the mobilisation of iron in Arabidopsis Tsai H H, Rodríguez-Celma J, Lan P, Wu Y C, Vélez-Bermúdez I C, et al. Scopoletin 8-hydroxylase-mediated fraxetin production is crucial for iron mobilization. Plant Physiol 2018; 177:194-207).

Sideretin is another catecholic coumarin also related to iron deficiency response and facilitates the uptake of iron in Arabidopsis. The 5′ hydroxylation of fraxetin to sideretin in Arabidopsis is catalysed by a cytochrome P450 enzyme termed CYP82C4. Apart from the compounds role in iron deficiency role in Arabidopsis—to the best of our knowledge—not much else is known about its functions (Rajniak J, Giehl R F H, Chang E, Murgia I, Von Wirén N, et al. Biosynthesis of redox-active metabolites in response to iron deficiency in plants. Nat Chem Biol 2018; 14:442-450; Robe K, Conejero G, Gao F, Lefebvre-Legendre L, Sylvestre-Gonon E, et al. Coumarin accumulation and trafficking in Arabidopsis thaliana: a complex and dynamic process. 2020. Epub ahead of print 2020. DOI: 10.1111/nph.17090).

It was thus the object of the invention to provide materials and methods to improve plant disease resistance, particularly in crops, and preferably also reducing the negative impact on overall plant health and/or yield which the means of obtaining said improved pathogen resistance may entail. In particular, it was a preferred object of the invention to provide materials and methods which lead to plant material of heritably improved resistance against fungal pathogens with minimised reduction of overall plant health, wherein resistance preferably is directed against a rust fungus and most preferably a fungus in the genus Phakopsora, Fusarium, Sclerotinia, Alternaria, Corynespora, Cercospora, or Septoria.

### SUMMARY OF THE INVENTION

The invention thus provides a plant cell capable of expressing a heterologous S8H and/or optionally a heterologous CYP82C4 enzyme.

In a further aspect the invention provides a plant or plant part comprising a plant cell capable of expressing a heterologous S8H and/or optionally a heterologous CYP82C4 enzyme.

In another aspect the invention provides a non-propagative plant part or material of a plant or plant part of the present invention, preferably a fermentation product, oil, meal, press cake, pomace, chaff, straw or compost.

In yet another aspect the invention provides a product of a plant, plant part or plant cell of the present invention, wherein the product is obtainable or obtained by
i) collecting a material of said plant, plant part or plant cell, preferably a harvestable plant part and most preferably a plant seed, and
ii) disrupting the collected material, preferably to obtain a fermentation product, oil, meal, press cake, pomace, chaff, straw or compost.

The invention also provides a method for improving plant health, comprising the step of conferring or increasing the production and/or accumulation of (a) fraxetin and optionally sideretin and/or of (b) a derivative thereof, in a plant cell, plant part or whole plant in comparison to a respective wild type plant, wild type plant part, or wild type plant cell.

In a further aspect the invention provides an automated plant selection method, comprising
i) obtaining, for each seed of a plurality of seeds, a sample comprising genetic material of a tissue body representative for said seed,
ii) determining the presence of a S8H and/or CYP82C4 gene as defined herein in the genetic material, and optionally the presence of one or more genes of a metabolic pathway for production of one or more coumarins,
iii) selecting those seed where the determination in step ii) gave a positive result.

And the invention provides a use of an S8H and/or CYP82C4 enzyme or of a nucleic acid comprising an expression cassette comprising an S8H gene and/or a CYP82C4 gene for any of:
i) conferring or increasing the production and/or accumulation of (a) fraxetin and optionally sideretin and/or of (b) a derivative thereof, in a plant cell, plant part or whole plant,
ii) improving plant health, preferably
modifying, reducing or removing plant health reducing effects of the production of esculetin, scopoletin and/or isoscopoletin
reducing, delaying or inhibiting germination or growth of a phytopathogenic microorganism on a surface of the plant or plant part, and/or
increasing resistance against infection by a phytopathogenic microorganism and/or increasing resistance against parasitic plants,
wherein the phytopathogenic microorganism preferably is selected from any of
phylum Ascomycota, Basidiomycota or Oomycota, more preferably of
order Pleosporales, Heliotiales, Hypocreales or Pucciniales, more preferably of
genus Alternaria, Botrytis, Sclerotinia, Fusarium, or, most preferred, Phakopsora.

### BRIEF DESCRIPTION OF THE DRAWINGS

FIG. 1: Schematic overview of sideretin biosynthesis. Feruloyl-CoA, derived from the phenylpropanoid pathway, is converted to scopoletin by adding hydroxyl-group at the 6′ C atom, followed by spontaneous isomerisation and lactonisation. The spontaneous reactions can also be catalysed by an enzyme termed COSY. The S8H further hydroxylates scopoletin at the 8′ C atom to form fraxetin. Subsequently, fraxetin is hydroxylated at the 5′ C atom, thereby forming sideretin. Through glucosylation the coumarins are converted into the storage form, likely accumulating in the vacuole. The active compound can again be released by hydrolysis catalysed by different BGLUs. F6′H1, Feruloyl-CoA 6′ hydroxylase 1; S8H, Scopoletin 8′ hydroxylase; CYP82C4, cytochrome P450 enzyme; UGT, UDP-Glc glucosyltransferase; BGLU, Betaglucosidase. Structural formulas were drawn with ChemDraw®.

FIG. 2: Overexpression of different combinations of coumarin-biosynthesis genes changes fluorescence. N. benthamiana leaves were transiently transformed using different overexpression constructs. (A) Three days after infiltration with Agrobacteria harbouring the overexpression constructs fluorescence was assessed under UV light. To adapt the picture to grey scale, green and blue fluorescence are depicted dark grey, whereas red chlorophyll fluorescence is shown in white. (B) Leaves were harvested and processed for qRT-PCR, where transcript abundance of overexpressed genes was quantified. Mean values and SD from two replicates.

FIG. 3: Transient overexpression of the three main biosynthesis genes yield sideretin. (A+B): Coumarins were extracted from N. benthamiana leaves transiently transformed with either F6′H1+S8H (A) or F6′H1+S8H+CYP82C4 (B) and subsequently separated via HPLC. The absorption at 342 nm was measured in the photodiode array, resulting in the depicted chromatograms. (C) The peak noted as sideretin was analysed in the photodiode array revealing the absorption spectrum between 230-500 nm.

FIG. 4: Accumulation of sideretin in transgenic soybean plants leads to increased resistance against soybean rust. The figure shows the result of the scoring of 59 transgenic soy plants derived from 5 independent transformation events (11-12 plants per event) expressing the sideretin producing gene combination S8H+CYP82C4 as described in example 7. The experiment was performed using plants of the T1 generation. Transgenicity of the plants was checked by PCR. Non-transgenic plants were discarded. T1 soybean plants harboring the sideretin producing gene combination cassette were inoculated with spores of Phakopsora pachyrhizi. The expression of the transgenes was checked by RT-PCR. The evaluation of the diseased leaf area was performed 14 days after inoculation. The average of the percentage of the leaf area showing fungal colonies or strong yellowing/browning on all leaves was considered as diseased leaf area. At all 59 soybean T1 plants accumulating sideretin by expressing the sideretin producing gene combination S8H+CYP82C4 were evaluated in parallel to non-transgenic control plants. The average of the diseased leaf area of S8H+CYP82C4 expressing plants and wild type control plants is shown in FIG. 4. Accumulation of Sideretin by expression of S8H+CYP82C4 significantly (**: p<0.01) reduces the diseased leaf area in comparison to non-transgenic control plants by 41%.

FIG. 5: A sequence alignment of SEQ ID NO. 1 (label: “SEQ1”) and the sequence according to Uniprot entry S8H_ARATH. Numbers are given according to the position of Uniprot entry S8H_ARATH sequence (label: “SEQ2”). The number of asterisks above each amino acid of the S8H_ARATH sequence indicates the degree of conservation, wherein higher number of stars indicate a stronger conservation. Amino acids given below each amino acid of the S8H_ARATH sequence are those of SEQ ID NO. 1, amino acids further below indicate potential substitutions allowable at the respective position, wherein “−” indicates a gap (deletion relative to the S8H_ARATH sequence). The possible substitutions are listed in the order of their respective preference, wherein a more preferred substitution is indicated closer to the respective position in SEQ ID NO. 2.

FIG. 6: A sequence alignment of SEQ ID NO. 3 (label: “SEQ3”) and the sequence according to Uniprot entry C82C4_ARATH. Numbers are given according to the position of Uniprot entry C82C4_ARATH sequence (label: “SEQ4”). The number of asterisks above each amino acid of the C82C4_ARATH sequence indicates the degree of conservation, wherein higher number of stars indicate a stronger conservation. Amino acids given below each amino acid of the C82C4_ARATH sequence are those of SEQ ID NO. 3, amino acids further below indicate potential substitutions allowable at the respective position, wherein “−” indicates a gap (deletion relative to the C82C4_ARATH sequence). The possible substitutions are listed in the order of their respective preference, wherein a more preferred substitution is indicated closer to the respective position in SEQ ID NO. 4.

FIG. 7: Nucleic acid sequences of genes coding for S8H_ARATH and C82C4_ARATH, respectively.

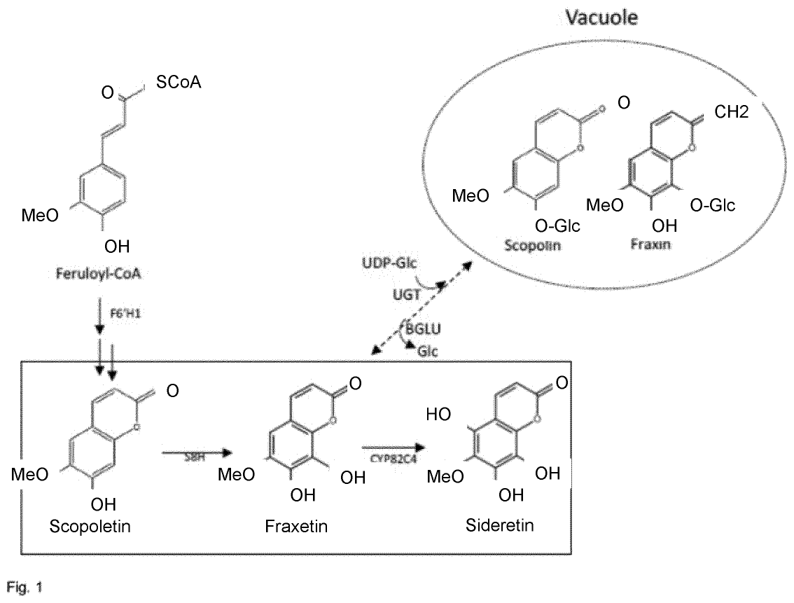

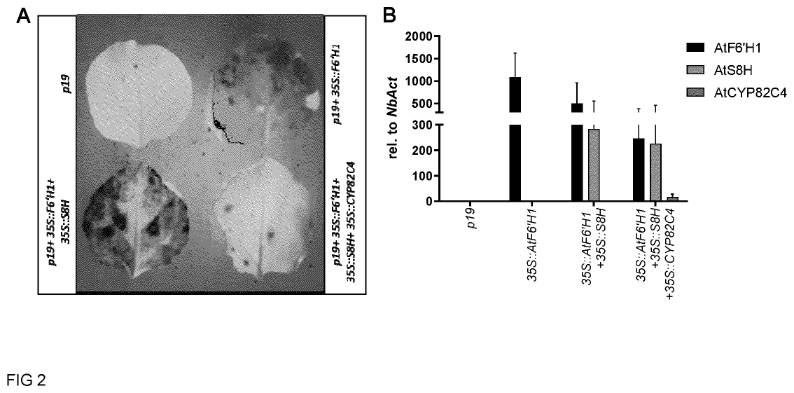

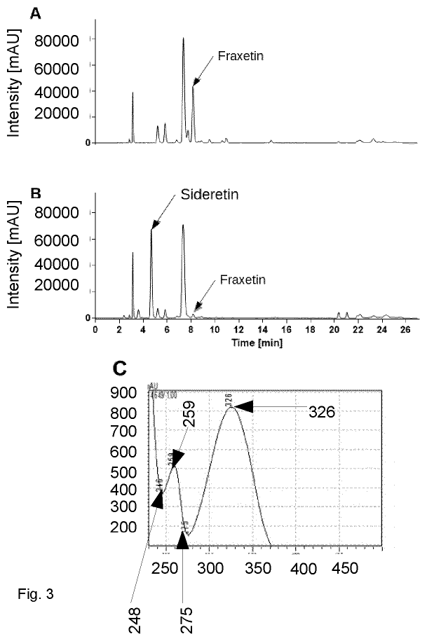

BRIEF DESCRIPTION OF THE SEQUENCES

| SEQ ID NO. | Description |
|---|---|
| 1 | sequence alignment template of an S8H protein sequence |
| 2 | Uniprot sequence S8H_ARATH |
| 3 | sequence alignment template of a CYP82C4 protein |
| | sequence |
| 4 | Uniprot sequence C82C4_ARATH |
| 5 | nucleotide sequence of a gene coding for SEQ ID NO. 2 |
| 6 | nucleotide sequence of a gene coding for SEQ ID NO. 4 |

### DETAILED DESCRIPTION OF THE INVENTION

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind the skilled person understands that the subject matter of the invention is the sum of the individual technical concepts signified herein or expressed, necessarily in a pars-pro-toto way, by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention.

In so far as recourse herein is made to entries in public databases, for example Uniprot, InterPro and PFAM, the contents of these entries are those as of 2022 Feb. 1. Unless stated to the contrary, where the entry comprises a nucleic acid or amino acid sequence information, such sequence information is incorporated herein.

As used herein, terms in the singular and the singular forms like “a”, “an” and “the” include plural referents unless the content clearly dictates otherwise. Thus, for example, use of the term “a nucleic acid” optionally includes, as a practical matter, many copies of that nucleic acid molecule; similarly, the term “probe” optionally (and typically) encompasses many similar or identical probe molecules. Also as used herein, the word “comprising” or variations such as “comprises” or “comprising” will be understood to imply the inclusion of a stated element, integer or step, or group of elements, integers or steps, but not the exclusion of any other element, integer or step, or group of elements, integers or steps.

As used herein, the term “and/or” refers to and encompasses any and all possible combinations of one or more of the associated listed items, as well as the lack of combinations when interpreted in the alternative (“or”). The term “comprising” also encompasses the term “consisting of”.

The term “about”, when used in reference to a measurable value, for example an amount of mass, dose, time, temperature, sequence identity and the like, refers to a variation of ±0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50% X whilst in other embodiments it may comprise anywhere from 40% to 60% X (i.e., 50%±10%).

As used herein, the term “gene” refers to a biochemical information which, when materialised in a nucleic acid, can be transcribed into a gene product, i.e. a further nucleic acid, preferably an RNA, and preferably also can be translated into a peptide or polypeptide. The term is thus also used to indicate the section of a nucleic acid resembling said information and to the sequence of such nucleic acid (herein also termed “gene sequence”).

Also as used herein, the term “allele” refers to a variation of a gene characterized by one or more specific differences in the gene sequence compared to the wild type gene sequence, regardless of the presence of other sequence differences. Alleles or nucleotide sequence variants of the invention have at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% nucleotide “sequence identity” to the nucleotide sequence of the wild type gene. Correspondingly, where an “allele” refers to the biochemical information for expressing a peptide or polypeptide, the respective nucleic acid sequence of the allele has at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% amino acid “sequence identity” to the respective wild type peptide or polypeptide.

“Substitutions” are described by providing the original amino acid followed by the number of the position within the amino acid sequence, followed by the substituted amino acid. For example, the substitution of histidine at position 120 with alanine is designated as “His120Ala” or “H120A”.

“Deletions” are described by providing the original amino acid followed by the number of the position within the amino acid sequence, followed by “−”. Accordingly, the deletion of glycine at position 150 is designated as “Gly150-” or “G150-”. Alternatively, deletions are indicated by e.g. “deletion of D183 and G184”.

“Terminations” are described by providing the original amino acid followed by the number of the position within the amino acid sequence, followed by “*”. Accordingly, an amino acid chain termination at position 150 instead of a glycine at this position is designated as “Gly150*” of “G150*”.

“Insertions” are described by providing the original amino acid followed by the number of the position within the amino acid sequence, followed by the original amino acid and the additional amino acid. For example, an insertion at position 180 of lysine next to glycine would be designated as “Gly180GlyLys” or “G180GK”. When more than one amino acid residue is inserted, such as e.g. a Lys and Ala after Gly180 this may be indicated as: Gly180GlyLysAla or G180GKA. In cases where a substitution and an insertion occur at the same position, this may be indicated as S99SD+S99A or in short S99AD. In cases where an amino acid residue identical to the existing amino acid residue is inserted, it is clear that degeneracy in the nomenclature arises. If for example a glycine is inserted after the glycine in the above example this would be indicated by G180GG. Furthermore, insertions can be described in the form of “−” followed by the position and the inserted amino acid, wherein the position is according to the sequence comprising the inserted amino acid. When aligning the sequences that do not and that do comprise the insertion, there will be a gap, indicated by “−”, at the position of insertion in the sequence that does not comprise the inserted amino acid.

Variants comprising multiple alterations are separated by “+”, e.g., “Arg170Tyr+Gly195Glu” or “R170Y+G195E” representing a substitution of arginine and glycine at positions 170 and 195 with tyrosine and glutamic acid, respectively. Alternatively, multiple alterations may be separated by space or a comma, e.g., R170Y G195E or R170Y, G195E respectively.

Where different alterations can be introduced at a position, the different alterations are separated by a comma, e.g., “Arg170Tyr, Glu” represents a substitution of arginine at position 170 with tyrosine or glutamic acid. Alternatively, different alterations or optional substitutions may be indicated in brackets e.g. Arg170[Tyr, Gly] or Arg170{Tyr, Gly} or in short R170[Y, G] or R170{Y, G}.

A special aspect concerning amino acid substitutions are conservative mutations which often appear to have a minimal effect on protein folding resulting in substantially maintained peptide or polypeptide properties of the respective peptide or polypeptide variant compared to the peptide or polypeptide properties of the parent peptide or polypeptide. Conservative mutations are those where one amino acid is exchanged with a similar amino acid. For determination of %-similarity the following applies, which is also in accordance with the BLOSUM62 matrix, which is one of the most used amino acids similarity matrix for database searching and sequence alignments:
Amino acid A is similar to amino acids S
Amino acid D is similar to amino acids E, N
Amino acid E is similar to amino acids D, K and Q
Amino acid F is similar to amino acids W, Y
Amino acid H is similar to amino acids N, Y
Amino acid I is similar to amino acids L, M and V
Amino acid K is similar to amino acids E, Q and R
Amino acid L is similar to amino acids I, M and V
Amino acid M is similar to amino acids I, L and V
Amino acid N is similar to amino acids D, H and S
Amino acid Q is similar to amino acids E, K and R
Amino acid R is similar to amino acids K and Q
Amino acid S is similar to amino acids A, N and T
Amino acid T is similar to amino acids S
Amino acid V is similar to amino acids I, L and M
Amino acid W is similar to amino acids F and Y
Amino acid Y is similar to amino acids F, H and W

Conservative amino acid substitutions may occur over the full length of the sequence of a polypeptide sequence of a functional protein such as a peptide or polypeptide. Preferably such mutations are not pertaining the functional domains of a peptide or polypeptide.

Protein or nucleic acid variants may be defined by their sequence identity when compared to a parent protein or nucleic acid. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:

Seq A:
AAGATACTG length: 9 bases
Seq B:
GATCTGA length: 7 bases

Hence, the shorter sequence is sequence B.

Producing a pairwise global alignment which is showing both sequences over their complete lengths results in

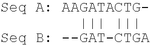

The “|” symbol in the alignment indicates identical residues (which means bases for DNA or amino acids for proteins). The number of identical residues is 6.

The “-” symbol in the alignment indicates gaps. The number of gaps introduced by alignment within the sequence B is 1. The number of gaps introduced by alignment at borders of sequence B is 2, and at borders of sequence A is 1.

The alignment length showing the aligned sequences over their complete length is 10.

Producing a pairwise alignment which is showing the shorter sequence over its complete length according to the invention consequently results in:

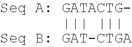

Producing a pairwise alignment which is showing sequence A over its complete length according to the invention consequently results in:

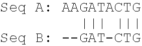

Producing a pairwise alignment which is showing sequence B over its complete length according to the invention consequently results in:

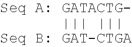

The alignment length showing the shorter sequence over its complete length is 8 (one gap is present which is factored in the alignment length of the shorter sequence).

Accordingly, the alignment length showing sequence A over its complete length would be 9 (meaning sequence A is the sequence of the invention), the alignment length showing sequence B over its complete length would be 8 (meaning sequence B is the sequence of the invention).

After aligning the two sequences, in a second step, an identity value shall be determined from the alignment. Therefore, according to the present description the following calculation of percent-identity applies:

%-identity=(identical residues/length of the alignment region which is showing the respective sequence of this invention over its complete length)*100. Thus, sequence identity in relation to comparison of two amino acid sequences according to the invention is calculated by dividing the number of identical residues by the length of the alignment region which is showing the respective sequence of this invention over its complete length. This value is multiplied with 100 to give “%-identity”. According to the example provided above, %-identity is: for sequence A being the sequence of the invention (6/9)*100=66.7%; for sequence B being the sequence of the invention (6/8)*100=75%.

The term “nucleic acid construct” as used herein refers to a nucleic acid molecule, either single- or double-stranded, which is isolated from a naturally occurring gene or is modified to contain segments of nucleic acids in a manner that would not otherwise exist in nature or is synthetic.

The term “nucleic acid construct” is synonymous with the term “expression cassette” when the nucleic acid construct contains the control sequences required for expression of a polynucleotide.

The term “control sequence” or “genetic control element” is defined herein to include all sequences affecting the expression of a polynucleotide, including but not limited thereto, the expression of a polynucleotide encoding a polypeptide. Each control sequence may be native or foreign to the polynucleotide or native or foreign to each other. Such control sequences include, but are not limited to, promoter sequence, 5′-UTR (also called leader sequence), ribosomal binding site (RBS), 3′-UTR, and transcription start and stop sites.

The term “functional linkage” or “operably linked” with respect to regulatory elements is to be understood as meaning the sequential arrangement of a regulatory element (including but not limited thereto a promoter) with a nucleic acid sequence to be expressed and, if appropriate, further regulatory elements (including but not limited thereto a terminator) in such a way that each of the regulatory elements can fulfil its intended function to allow, modify, facilitate or otherwise influence expression of said nucleic acid sequence. For example, a control sequence is placed at an appropriate position relative to the coding sequence of the polynucleotide sequence such that the control sequence directs the expression of the coding sequence of a polypeptide.

A “promoter” or “promoter sequence” is a nucleotide sequence located upstream of a gene on the same strand as the gene that enables that gene's transcription. A promoter is generally followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

As used herein, the term “isolated DNA molecule” refers to a DNA molecule at least partially separated from other molecules normally associated with it in its native or natural state. The term “isolated” preferably refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods well known to those skilled in the art can be used to isolate and manipulate a polynucleotide, or fragment thereof, as disclosed herein. For example, polymerase chain reaction (PCR) technology can be used to amplify a particular starting polynucleotide molecule and/or to produce variants of the original molecule. Polynucleotide molecules, or fragment thereof, can also be obtained by other techniques, such as by directly synthesizing the fragment by chemical means, as is commonly practiced by using an automated oligonucleotide synthesizer. A polynucleotide can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments of the method include those wherein the polynucleotide is at least one selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used.

The term “heterologous” means that the respective genetic element does not naturally occur in wild type cells. Thus, for example, a heterologous enzyme is an enzyme such that no gene in the corresponding wild type cell codes for a protein with the same sequence as the heterologous enzyme.

As used herein, “recombinant” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if (a) that nucleotide sequence is present in a context other than its natural one, for example by virtue of being (i) cloned into any type of artificial nucleic acid vector or (ii) moved or copied to another location of the original genome, or if (b) the nucleotide sequence is mutagenized such that it differs from the wild type sequence. The term recombinant also can refer to an organism having a recombinant material, e.g., a plant that comprises a recombinant nucleic acid is a recombinant plant.

The term “transgenic” refers to an organism, preferably a plant or part thereof, or a nucleic acid that comprises a heterologous polynucleotide. Preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been so altered by the presence of heterologous nucleic acid including those transgenic organisms or cells initially so altered, as well as those created by crosses or asexual propagation from the initial transgenic organism or cell. A “recombinant” organism preferably is a “transgenic” organism. The term “transgenic” as used herein is not intended to encompass the alteration of the genome (chromosomal or extra-chromosomal) by conventional plant breeding methods (e.g., crosses) or by naturally occurring events such as, e.g., self-fertilization, random cross-fertilization, non-recombinant viral infection, non-recombinant bacterial transformation, non-recombinant transposition, or spontaneous mutation.

As used herein, “mutagenized” refers to an organism or nucleic acid thereof having alteration(s) in the biomolecular sequence of its native genetic material as compared to the sequence of the genetic material of a corresponding wildtype organism or nucleic acid, wherein the alteration(s) in genetic material were induced and/or selected by human action. Examples of human action that can be used to produce a mutagenized organism or DNA include, but are not limited to treatment with a chemical mutagen such as EMS and subsequent selection with herbicide(s); or by treatment of plant cells with x-rays and subsequent selection with herbicide(s). Any method known in the art can be used to induce mutations. Methods of inducing mutations can induce mutations in random positions in the genetic material or can induce mutations in specific locations in the genetic material (i.e., can be directed mutagenesis techniques), such as by use of a genioplasty technique. In addition to unspecific mutations, according to the invention a nucleic acid can also be mutagenized by using mutagenesis means with a preference or even specificity for a particular site, thereby creating an artificially induced heritable allele according to the present invention. Such means, for example site specific nucleases, including for example zinc finger nucleases (ZFNs), meganucleases, transcription activator-like effector nucleases (TALENS) (Malzahn et al., Cell Biosci, 2017, 7:21) and clustered regularly interspaced short palindromic repeats/CRISPR-associated nuclease (CRISPR/Cas) with an engineered crRNA/tracr RNA (for example as a single-guide RNA, or as modified crRNA and tracrRNA molecules which form a dual molecule guide), and methods of using this nucleases to target known genomic locations, are well known in the art (see reviews by Bortesi and Fischer, 2015, Biotechnology Advances 33:41-52; and by Chen and Gao, 2014, Plant Cell Rep 33:575-583, and references within).

As used herein, a “genetically modified organism” (GMO) is an organism whose genetic characteristics contain alteration(s) that were produced by human effort causing transfection that results in transformation of a target organism with genetic material from another or “source” organism, or with synthetic or modified-native genetic material, or an organism that is a descendant thereof that retains the inserted genetic material. The source organism can be of a different type of organism (e.g., a GMO plant can contain bacterial genetic material) or from the same type of organism (e.g., a GMO plant can contain genetic material from another plant).

As used herein, “wildtype” or “corresponding wildtype plant” means the typical form of an organism or its genetic material, as it normally occurs, as distinguished from e.g. mutagenized and/or recombinant forms. Similarly, by “control cell”, “wildtype” “control plant, plant tissue, plant cell or host cell” is intended a plant, plant tissue, plant cell, or host cell, respectively, that lacks the particular polynucleotide of the invention that are disclosed herein. The use of the term “wildtype” is not, therefore, intended to imply that a plant, plant tissue, plant cell, or other host cell lacks recombinant DNA in its genome, and/or does not possess fungal resistance characteristics that are different from those disclosed herein.

As used herein, “descendant” refers to any generation plant. A progeny or descendant plant can be from any filial generation, e.g., F1, F2, F3, F4, F5, F6, F7, etc. In some embodiments, a descendant or progeny plant is a first, second, third, fourth, fifth, sixth, seventh, eighth, ninth, or tenth generation plant.

The term “plant” is used herein in its broadest sense as it pertains to organic material and is intended to encompass eukaryotic organisms that are members of the taxonomic kingdom plantae, examples of which include but are not limited to monocotyledon and dicotyledon plants, vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, underground stems, clumps, crowns, bulbs, corms, tubers, rhizomes, plants/tissues produced in tissue culture, etc.). Unless stated otherwise, the term “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A plant cell is a biological cell of a plant, taken from a plant or derived through culture from a cell taken from a plant.

The invention particularly applies to plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato or tobacco. The plant preferably is not of taxonomic family Solanaceae, more preferably not of sub-family Solanoidae.

According to the invention, a plant is cultivated to yield plant material. Cultivation conditions are chosen in view of the plant and may include, for example, any of growth in a greenhouse, growth on a field, growth in hydroculture and hydroponic growth.

It is to be understood that when reference is made to advantages conferred by of “a plant” or by a plant part or cell, it is preferred not to determine the advantages on a single plant compared to a single control plant. Instead, advantages are apparent by inspecting an ensemble of the plants, preferably an ensemble of at least 1000 plants, preferably wherein the plants are cultivated on a field or, less preferably, in a greenhouse. Most preferably the plants are grown on a monoculture field of at least 1 ha of the plant and a monoculture field of at least 1 ha of the control plant is determined, respectively. Correspondingly, treatments are preferably performed on such ensemble of plants. Furthermore, references to harvestable plant material, preferably of fruit and seed, refer to at least 1000 kg (drained weight) of such material, preferably a fill of 1000-10000000 kg.

The invention provides, in particular, means for increasing the production or accumulation of fraxetin, and preferably also sideretin, in coumarin-producing cells, notably those that produce scopoletin. It has now been surprisingly found that plant health adversarial effects of scopoletin production, in particular low yield, stunted or slow growth, low vigor and a generally unhealthy phenotype, such as yellowing of leaves and early leaf drop can be significantly reduced by conversion of scopoletin into fraxetin or derivatives thereof, e.g. sideretin or fraxin. Thus, the present invention overcomes a major obstacle that prevented the use of artificial coumarin production in planta which would otherwise be a useful tool to prevent or reduce attacks by plant pathogens.

Correspondingly the invention provides plant cell capable of expressing a heterologous S8H enzyme. The S8H enzyme belongs, in InterPro nomenclature, to the IPR027443 superfamily. One way to describe its structure is that the enzyme comprises, again in InterPro nomenclature and in N- to C-terminal direction, (i) an IPR026992 non-haem dioxygenase N-terminal domain and (ii) an IPR005123 oxoglutarate/iron-dependent dioxygenase domain and/or an IPR044861 isopenicillin N synthase-like Fe(2+) 2OG dioxygenase domain.

Furthermore, the invention also provides plant cells capable of expressing a heterologous CYP82C4 enzyme. The CYP82C4 enzyme belongs, in InterPro nomenclature, to the IPR0363696 superfamily. One way to describe its structure is that the enzyme comprises, again in InterPro nomenclature, an IPR001128 cytochrome P560 domain.

In accordance with the general teaching of the invention it has now been found that the conversion of scopoletin into fraxetin and/or sideretin or derivatives thereof can be facilitated by providing a heterologous S8H and/or CYP82C4 enzyme in plant cells, thereby reducing plant health adversarial effects of coumarin synthesis while maintaining or even increasing pest resistance of the plant comprising such cells.

Preferably the S8H enzyme amino acid sequence has 30-90% identity to SEQ ID NO. 1, preferably 50-75% identity, more preferably 70-74% sequence identity. Also preferably the CYP82C4 enzyme amino acid sequence has 34-90% identity to SEQ ID NO. 3, preferably 60-75%, more preferably 66-74%. It is to be noted that both SEQ ID NO. 1 and SEQ ID NO. 3 do not refer to a protein sequence whose enzymatic activity has been ascertained. Instead, the sequence has been artificially constructed as a template to find, by sequence alignment, corresponding further S8H or CYP82C4 protein sequences. Thus, the maximum sequence identity of a functional S8H or CYP82C4 enzyme sequence will be less than 100%, and indeed is at most 90%.

Preferably the gene coding for the S8H enzyme is obtainable or obtained by selecting, from the genome of a plant of clade Gunneridae, a gene coding for a protein having 30-90% identity to SEQ ID NO. 1, preferably 50-75% identity, more preferably 70-74% sequence identity, and/or having 69-100% identity to SEQ ID NO. 2, preferably 83-100%. More preferably the gene is selected from a plant of genus Arabidopsis, Capsella, Brassica, Theobroma, Arabis, Microthlaspi, Raphanus, Actinidia, Arachis, Carpinus, Durio, Herrania, Artemisia, Malus, Glycine, Corchorus, Gossypium, Cajanus, Hevea, Phaseolus, Vigna, Citrus, Salix, Jatropha, Hibiscus, Nyssa, Populus, Acer, Morus, Manihot, Lactuca, Coffea, Lupinus, Helianthus, Vitis, Daucus, Nicotiana, Prunus, Solanum, Mikania, Eutrema, Ricinus, Quercus, Thalictrum, Pyrus, Parasponia, Trema, Juglans, Cannabis, Camellia or Macleaya, even more preferably, in decreasing order of preference, of genus Arabidopsis, Capsella, Brassica, Theobroma, Arabis, Microthlaspi, Raphanus, Actinidia, Arachis, Carpinus, Durio, Herrania, Artemisia, Malus, Glycine, Corchorus, Gossypium, Cajanus, Hevea, Phaseolus, Vigna, Citrus, Salix, Jatropha, Hibiscus, Nyssa, Populus, Acer, Morus, Manihot, Lactuca, Coffea, Lupinus, Helianthus, Vitis, Daucus, Nicotiana, Prunus, Solanum, Mikania, Eutrema or Ricinus, even more preferably of family Brassicaceae, even more preferably, in decreasing order or preference, of genus Arabidopsis, Arabis, Microthlaspi, Brassica, Raphanus, Eutrema or Capsella, even more preferably of any of the species Arabidopsis thaliana, Arabidopsis lyrata, Arabis alpina, Arabis nemorensis, Brassica campestris, Brassica napus, Brassica oleracea, Brassica rapa, Capsella rubella, Eutrema salsugineum, Microthlaspi erraticum or Raphanus sativus and most preferred of genus Arabidopsis. Preferred S8H enzyme sequences are found under the following Uniprot identifiers, in decreasing order of preference: S8H_ARATH, D7L0L4_ARALL, A0A565B701_9BRAS, A0A087H930_ARAAL, A0A6D2HJZ6_9BRAS, A0A398A866_BRACM, M4CB96_BRARP, A0A0D3BB07_BRAOL, A0A0D3CKR8_BRAOL, A0A078JXY8_BRANA, A0A6J0JSE1_RAPSA, A0A078J1U2_BRANA, A0A078IYI9_BRANA, A0A6J0P048_RAPSA, A0A6J0LWT5_RAPSA, A0A397ZKJ4_BRACM, A0A078F5S4_BRANA, M4FE31_BRARP, A0A0D3AE45_BRAOL, A0A078GFX9_BRANA, M4F0X5_BRARP, A0A398AYA5_BRACM, V4M1N3_EUTSA, A0A2C9UIT2_MANES, A0A6P6BIN7_DURZI, V4S9D6_CITCL, A0A2P5WEH0_GOSBA, A0A5D2W349_GOSMU, A0A1U8M084_GOSHI, A0A5D3ACU4_GOSMU, A0A5B6VFL1_9ROSI, A0A067FEL4_CITSI, A0A0D2Q0A8_GOSRA, A0A6P5WP01_DURZI, A0A6P5WQF3_DURZI, A0A2R6Q7J8_ACTCC, A0A6J1AL44_9ROSI, A0A1U8MBX2_GOSHI, A0A6P5Y1H7_DURZI, A0A6P5WNB1_DURZI, A0A6P5WQV9_DURZI, A0A6P5WMV5_DURZI, A0A067JYM9_JATCU, A0A5N5MR48_9ROSI, A0A5D2W3A7_GOSMU, A0A061FAM3_THECC, A0A1R3G5V5_9ROSI, A0A5J5ADB0_9ASTE, A0A1R3KY58_9ROSI, F6H4S9_VITVI, A0A5D3ABE5_GOSMU, A0A0D2R079_GOSRA, A0A2R6Q7F1_ACTCC, A0A6J1ALF8_9ROSI, A0A6A4N9J3_LUPAL, A0A5B6VF76_9ROSI, A0A6J1AM19_9ROSI, D7SZ13_VITVI, D7SZ09_VITVI, A0A6P6BIR0_DURZI, A0A5D3AE44_GOSMU, A0A0D2Q0A6_GOSRA, A0A540NCJ2_MALBA, A0A067FDX9_CITSI, V4RMG0_CITCL, A0A6S7PA59_LACSI, A0A4U5QG74_POPAL, A0A1U8MBW6_GOSHI, A0A1U8M3T1_GOSHI, A0A2U1MCH7_ARTAN, B9H637_POPTR, A0A1R3G608_9ROSI, A0A5D3ACT5_GOSMU, A0A2P5Y6K1_GOSBA, A0A1R3G5S5_9ROSI, A0A6J1AML8_9ROSI, A0A6J1AK73_9ROSI, A0A5D2W2X0_GOSMU, A0A498JX31_MALDO, A0A5C7IQ36_9ROSI, A0A0B0MWP3_GOSAR, A0A5C7IS84_9ROSI, A0A6A3AH12_HIBSY, A0A2P5Y6L4_GOSBA, A0A5N6RT17_9ROSI, A0A078HZT3_BRANA, A0A314Y0W6_PRUYE, A0A2P5Y6M0_GOSBA, M5WMS7_PRUPE, A0A6J1ALC3_9ROSI, A0A1U8M068_GOSHI, A0A5C7IL41_9ROSI, A0A6P5WPZ2_DURZI, A0A445JFQ3_GLYSO, I1KU12_SOYBN, A0A2P5WEG3_GOSBA, A0A6P6SIX2_COFAR, A0A5C7IPK0_9ROSI, A0A2J6JVJ9_LACSA, A0A6P5TNE2_PRUAV, A0A6P6VF23_COFAR, A0A6A2YWM8_HIBSY, R0HTK9_9BRAS, A0A444XPT9_ARAHY, A0A068V9Q2_COFCA, A0A061FAL9_THECC, A0A5E4FJW9_PRUDU, A0A5C7IPL6_9ROSI, A0A5B6VFW0_9ROSI, A0A5D2W2G0_GOSMU, A0A6A6MHB3_HEVBR, A0A6A3ATA3_HIBSY, A0A2C9VT53_MANES, A0A1U8MC28_GOSHI, A0A0D2NHU3_GOSRA, A0A5B6W5I7_9ROSI, A0A067JZY8_JATCU, A0A165Y8S4_DAUCS, A0A6P4BIR7_ARADU, A0A445BIE6_ARAHY, A0A1U7WQN2_NICSY, A0A1S3XN71_TOBAC, A0A0L9UJA1_PHAAN, A0A0S3SK96_PHAAN, A0A6P6T6W1_COFAR, A0A2C9UIS4_MANES, A0A6A3AFK6_HIBSY, A0A151R218_CAJCA, A0A1S3U4E3_VIGRR, A0A2U1N397_ARTAN, A0A6A4R6E2_LUPAL, W9R0E9_9ROSA, A0A061FH15_THECC, A0A1S4CRF9_TOBAC, A0A068VHC0_COFCA, A0A4P1R936_LUPAN, V7BW78_PHAVU, A0A2U1QIJ9_ARTAN, A0A5D2SZC1_GOSMU, A0A0D2TQV4_GOSRA, A0A251UL64_HELAN, A0A1U8HHI8_GOSHI, A0A3Q7IWJ1_SOLLC, A0A314LF54_NICAT, A0A6P5WPN5_DURZI, A0A6A3AKC1_HIBSY, A0A1R3G5V1_9ROSI, A0A5N6M6G4_9ASTR, A0A6A3AKR7_HIBSY, A0A1U8MFJ1_GOSHI, A0A061F9P0_THECC, A0A2U1MCG7_ARTAN, A0A2J6JVL4_LACSA, A0A2H5NRT1_CITUN, B9RI27_RICCO, A0A2U1LUR0_ARTAN and A0A5B6VHC0_9ROSI, more preferably, again in decreasing order of preference, S8H_ARATH, D7L0L4_ARALL, A0A565B701_9BRAS, A0A087H930_ARAAL, A0A6D2HJZ6_9BRAS, A0A398A866_BRACM, M4CB96_BRARP, A0A0D3BB07_BRAOL, A0A0D3CKR8_BRAOL, A0A078JXY8_BRANA, A0A6J0JSE1_RAPSA, A0A078J1U2_BRANA, A0A078IYI9_BRANA, A0A6J0P048_RAPSA, A0A6J0LWT5_RAPSA, A0A397ZKJ4_BRACM, A0A078F5S4_BRANA, M4FE31_BRARP, A0A0D3AE45_BRAOL, A0A078GFX9_BRANA, M4F0X5_BRARP, A0A398AYA5_BRACM, V4M1N3_EUTSA, A0A078HZT3_BRANA and R0HTK9_9BRAS.

Preferably the S8H enzyme sequence differs from the sequence according to SEQ ID NO. 1 or SEQ ID NO. 2 only by conservative mutations. As described above, conservative mutations generally do not interfere with the structure of a protein, they have a high probability of maintaining the enzyme functional. Thus, for optimising the respective enzyme sequence it is advised to apply conservative mutations.

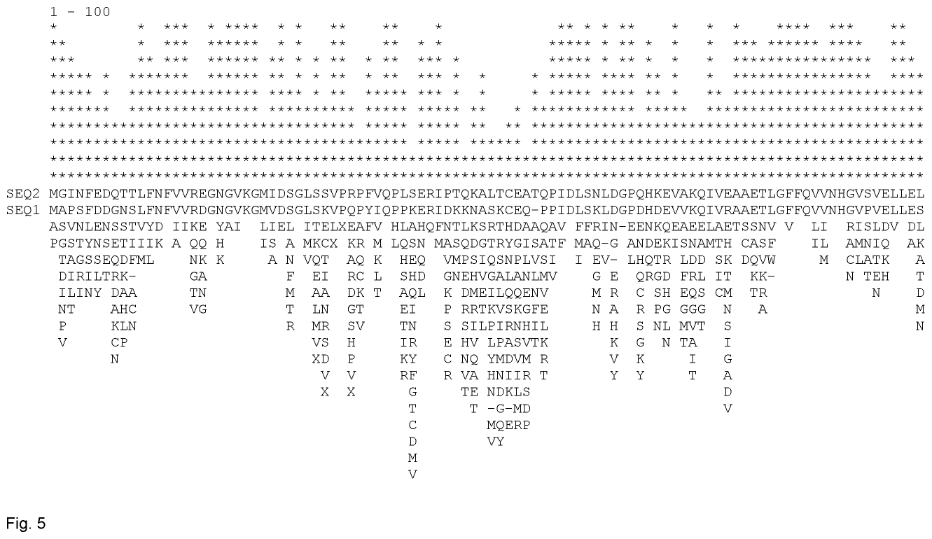

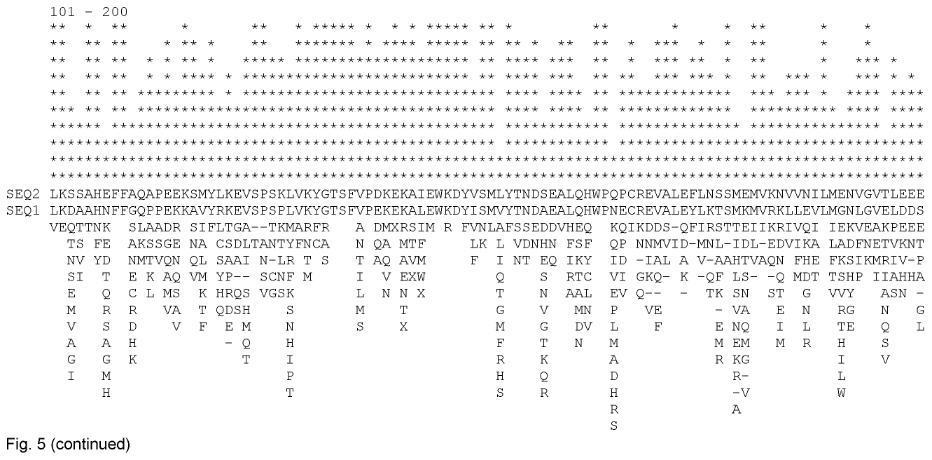

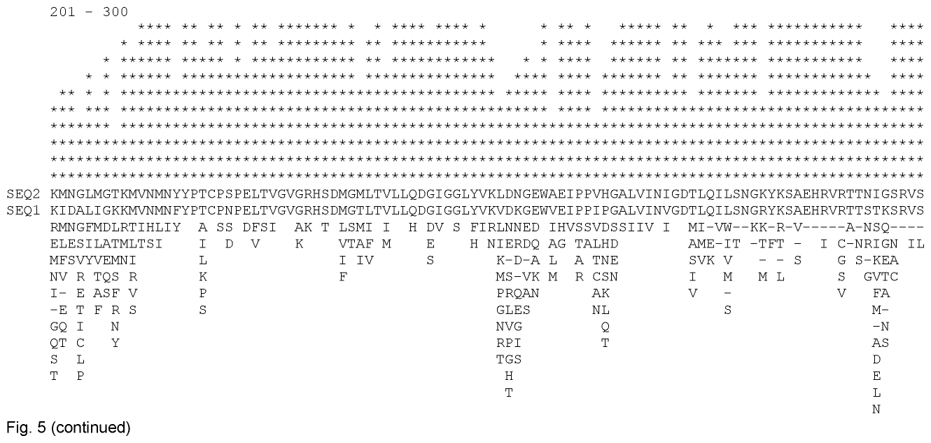

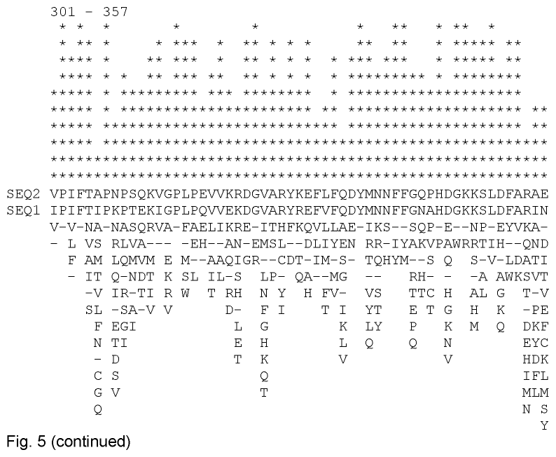

Even more preferably, the S8H enzyme sequence differs from the sequence according to SEQ ID NO. 1 only according to the amino acids given in FIG. 5 for each respective position. It has been found that these amino acids occur, at the respective positions, in homologous enzyme sequences. Thus, by limiting exchanges (substitutions, insertions or deletions) in for example a sequence according to one of the aforementioned Uniprot identifiers only to the sequences allowed according to this figure, the resulting mutant enzyme can have a restored or improved enzymatic activity.

Particularly preferred is an S8H enzyme having the sequence according to SEQ ID NO. 2, i.e. Uniprot identifier S8H_ARATH. As shown in the examples below such enzyme has been found to be functional and highly active, thereby allowing to achieve the advantages of the present invention. Accordingly, it is preferred that the S8H enzyme sequence only differs from SEQ ID NO. 1 by one, more or all of the following mutations, in the numbering according to SEQ ID NO. 2: A2G, P3I, S4N, D6E, G8Q, N9T, S10T, D18E, V26I, K32S, Q35R, Y37F, I38V, P41L, K42S, D46P, K47T, K48Q, N49K, S51L, K52T, Q55A, -56T, P57Q, K63N, D68Q, D70K, V73A, R78E, P94S, S100L, D103S, A104S, N107E, G110A, P112A, K115E, A117S, V118M, R120L, P127K, E138D, L143I, I149V, V152L, A157S, N165Q, E166P, Y174F, K176N, T177S, K180E, R183K, K184N, L185V, L186V, E187N, V188I, G191E, L193V, E196T, D198E, D199E, S200E, I202M, D203N, A204G, I206M, K208T, F215Y, N221S, T236M, V252L, K254N, V258A, I263V, P264H, V271I, R282K, S294N, T295I, K296G, I301V, I306A, K308N, T310S, E311Q, I313V, Q318E, E321K, K322R, R329K, V332L, N343Q, A344P, I356A, N357E. These mutations increase the identity between a candidate S8H enzyme sequence, e.g. according to one of the aforementioned Uniprot identifiers, and the most preferred sequence. Thus, such mutations provide a way to align a candidate enzyme sequence to the most preferred one and to improve its function, thereby achieving the advantages of the present invention.

Preferably the gene coding for the CYP82C4 enzyme is obtainable or obtained by selecting, from the genome of a plant of clade Gunneridae, a gene coding for a protein having 34-90% identity to SEQ ID NO. 3, preferably 60-75%, more preferably 66-74%, and/or having 70-100% identity to SEQ ID NO. 4, preferably 86-100%. More preferably the gene is selected from a plant of genus Arabidopsis, Capsella, Brassica, Theobroma, Arabis, Microthlaspi, Raphanus, Actinidia, Arachis, Carpinus, Durio, Herrania, Artemisia, Malus, Glycine, Corchorus, Gossypium, Cajanus, Hevea, Phaseolus, Vigna, Citrus, Salix, Jatropha, Hibiscus, Nyssa, Populus, Acer, Morus, Manihot, Lactuca, Coffea, Lupinus, Helianthus, Vitis, Daucus, Nicotiana, Prunus, Solanum, Mikania, Eutrema, Ricinus, Quercus, Thalictrum, Pyrus, Parasponia, Trema, Juglans, Cannabis, Camellia or Macleaya, even more preferably, in decreasing order of preference, of genus Arabidopsis, Brassica, Populus, Prunus, Solanum, Nicotiana, Lactuca, Citrus, Vigna, Arachis, Lupinus, Mikania, Microthlaspi, Raphanus, Artemisia, Hevea, Manihot, Ricinus, Arabis, Quercus, Helianthus, Gossypium, Hibiscus, Actinidia, Glycine, Durio, Thalictrum, Corchorus, Nyssa, Vitis, Herrania, Theobroma, Phaseolus, Morus, Malus, Pyrus, Parasponia, Trema, Juglans, Cannabis, Acer, Jatropha, Camellia, Salix, Cajanus, Macleaya or Daucus, even more preferably, in decreasing order of preference, any of genus Arabidopsis, Brassica, Populus, Prunus, Solanum, Nicotiana, Lactuca, Citrus, Vigna, Arachis, Lupinus, Mikania, Microthlaspi, Raphanus, Artemisia, Hevea, Manihot, Ricinus, Arabis, Quercus, Helianthus, Gossypium, Hibiscus, Actinidia, Glycine, Durio, Thalictrum, Corchorus, Nyssa, Vitis, Herrania, Theobroma, Phaseolus, Morus, Malus, Pyrus, Parasponia, Trema, Juglans, Cannabis, Acer, Jatropha, Camellia, Salix, Cajanus, Macleaya or Daucus, even more preferably of family Brassicaceae, even more preferably, in decreasing order or preference, of genus Arabidopsis, Arabis, Microthlaspi, Brassica or Raphanus, even more preferably of species Arabidopsis lyrata, Arabidopsis thaliana, Arabis alpina, Brassica campestris, Brassica napus, Brassica oleracea, Brassica rapa, Microthlaspi erraticum, Raphanus sativus and most preferred of genus Arabidopsis. Preferred CYP82C4 enzyme sequences are found under the following Uniprot identifiers, in decreasing order of preference: C82C4_ARATH, D7MAT6_ARALL, D7MAT3_ARALL, A0A087GIV7_ARAAL, A0A6D2JQ87_9BRAS, A0A078GD55_BRANA, A0A078IWE0_BRANA, C82C2_ARATH, A0A0D3BLW3_BRAOL, M4D165_BRARP, A0A397YAX0_BRACM, A0A6J0KT84_RAPSA, C82C3_ARATH, A0A2R6RN06_ACTCC, A0A5C7H6G1_9ROSI, A0A2K1XQ31_POPTR, A0A7N2KS04_QUELO, A0A7J9A9I0_9ROSI, A0A5B6X3U4_9ROSI, A0A1R3HX50_9ROSI, I1JTF2_SOYBN, A0A445KV13_GLYSO, A0A5J5AEA6_9ASTE, E0CPD8_VITVI, A0A7J8V8M2_9ROSI, A0A7J8TIT9_GOSDV, A0A1U8HP27_GOSHI, A0A6P4B9P2_ARADU, A0A397YCM3_BRACM, A0A445BRY3_ARAHY, A0A7J8MMD6_9ROSI, A0A5D2ZB63_GOSMU, A0A445CBK5_ARAHY, A0A2P5FMX7_TREOI, A0A061FCJ9_THECC, A0A2P5DA28_PARAD, A0A1U8MYF8_GOSHI, A0A0D2TT10_GOSRA, A0A5D2UZ32_GOSMU, A0A540MG66_MALBA, V4T9G8_CITCL, A0A6J1AEJ3_9ROSI, A0A498HT27_MALDO, A0A7J9LZH2_GOSSC, A0A0L9V973_PHAAN, A0A0S3RBD3_PHAAN, A0A1S3Z2V3_TOBAC, V7AVM1_PHAVU, A0A251RCK3_PRUPE, A0A6P5SX50_PRUAV, A0A6J5W4K7_PRUAR, A0A1J6K998_NICAT, A0A6P6B514_DURZI, A0A7J6DX77_CANSA, A0A5N5GVN6_9ROSA, W9S3T8_9ROSA, A0A6A2YBQ1_HIBSY, A0A2H5NGY5_CITUN, A0A2I4GS12_JUGRE, A0A2H5NH90_CITUN, A0A5E4E8D8_PRUDU, A0A7J9JS55_9ROSI, A0A1S3U046_VIGRR, A0A4P1RNQ9_LUPAN, A0A1S3ZVD4_TOBAC, K4DGW8_SOLLC, A0A7J6W7E4_THATH, A0A5N6PBL6_9ASTR, A0A2U1MHF3_ARTAN, A0A4U5QZH8_POPAL, A0A6A5M1C5_LUPAL, A0A2J6KSC2_LACSA, A0A6S7L551_LACSI, A0A6S7L543_LACSI, A0A2J6KS68_LACSA, A0A6A6NH10_HEVBR, A0A2C9VX22_MANES, B9R7K5_RICCO, A0A251VDS8_HELAN, A0A7J6DJB5_CANSA, A0A251UC93_HELAN, A0A5N6PD06_9ASTR, A0A2K1XQ11_POPTR, A0A6P5SVN5_PRUAV, A0A2J6JPW6_LACSA, A0A4U5QXJ6_POPAL, A0A1U7YD68_NICSY, A0A067KTW6_JATCU, M1B8W8_SOLTU, A0A151TC08_CAJCA, A0A2P5WH20_GOSBA, A0A200QK53_9MAGN, A0A4S4DSN8_CAMSI, A0A061FC95_THECC, A0A5N5KBD4_9ROSI, A0A161XZE2_DAUCS, A0A444WSS6_ARAHY, A0A1R3HX54_9ROSI, A0A251V9U3_HELAN and A0A7J9IE65_9ROSI, more preferably, again in decreasing order of preference, C82C4_ARATH, D7MAT6_ARALL, D7MAT3_ARALL, A0A087GIV7_ARAAL, A0A6D2JQ87_9BRAS, A0A078GD55_BRANA, A0A078IWE0_BRANA, C82C2_ARATH, A0A0D3BLW3_BRAOL, M4D165_BRARP, A0A397YAX0_BRACM, A0A6J0KT84_RAPSA, C82C3_ARATH and A0A397YCM3_BRACM.

Furthermore, the CYP82C4 enzyme sequence preferably differs from the sequence according to SEQ ID NO. 3 or SEQ ID NO. 4 only by conservative mutations. As described above, conservative mutations generally do not interfere with the structure of a protein, they have a high probability of maintaining the enzyme functional. Thus, for optimising the respective enzyme sequence it is advised to apply conservative mutations.

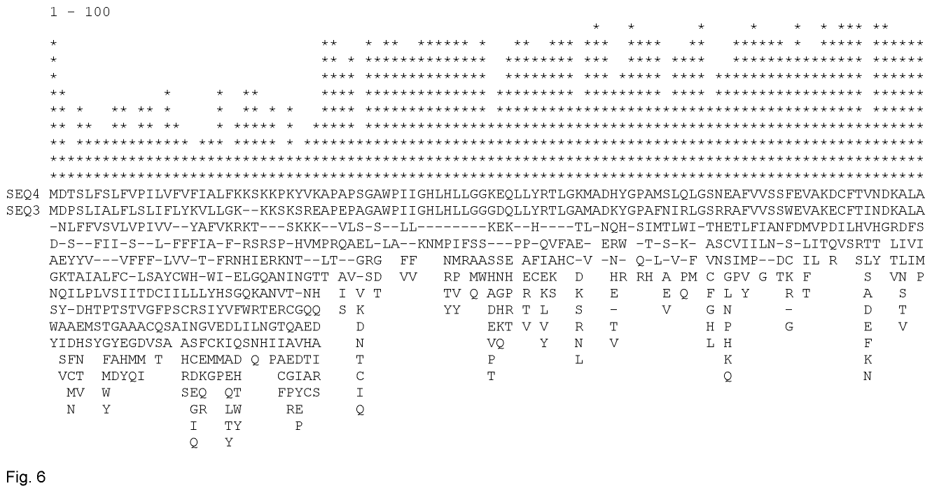

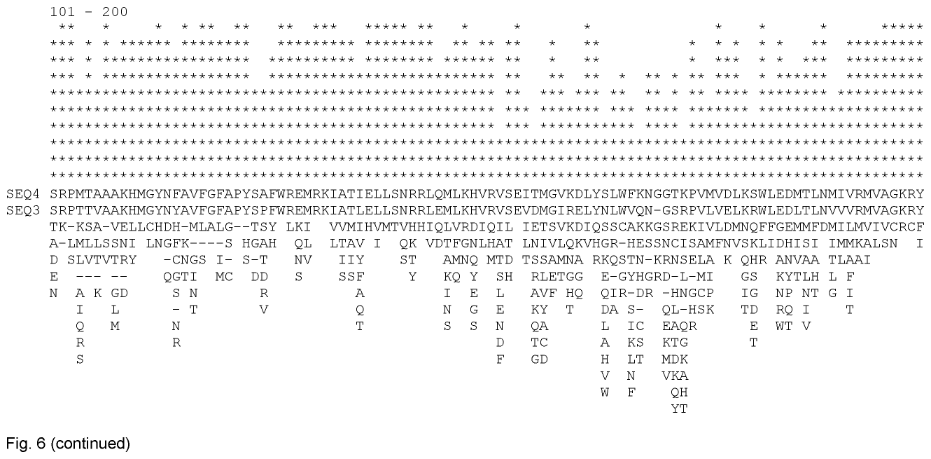

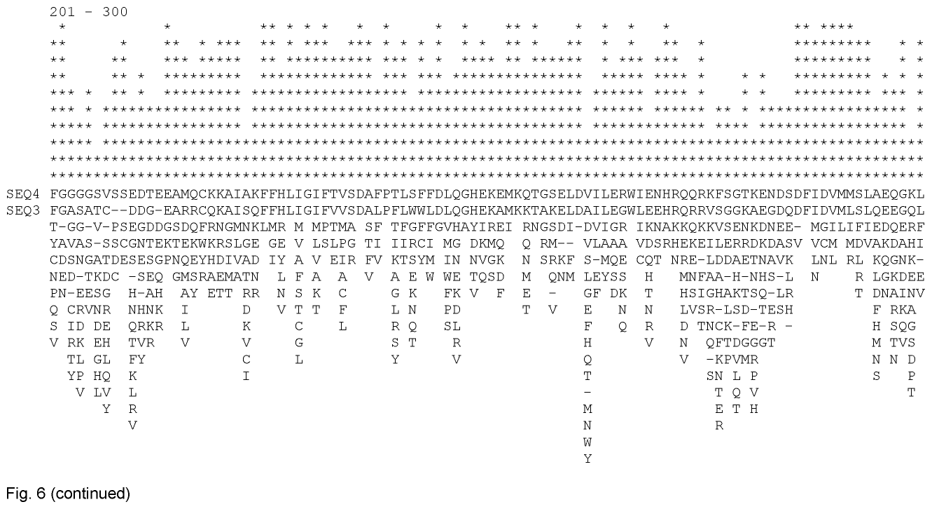

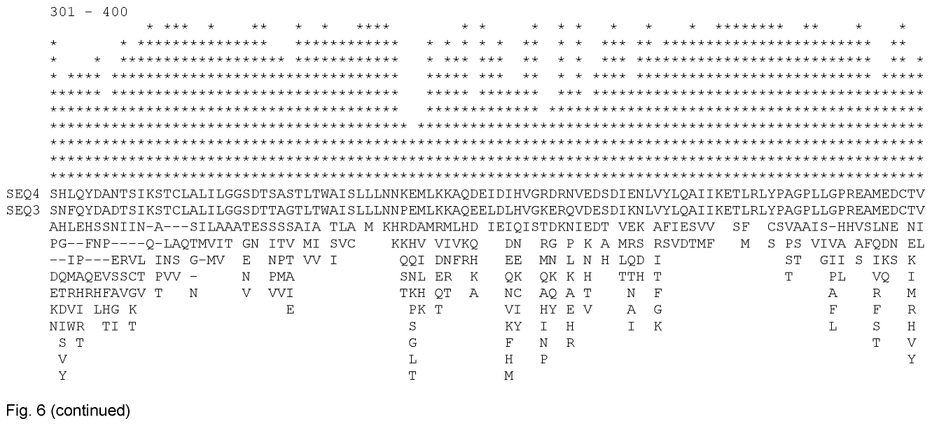

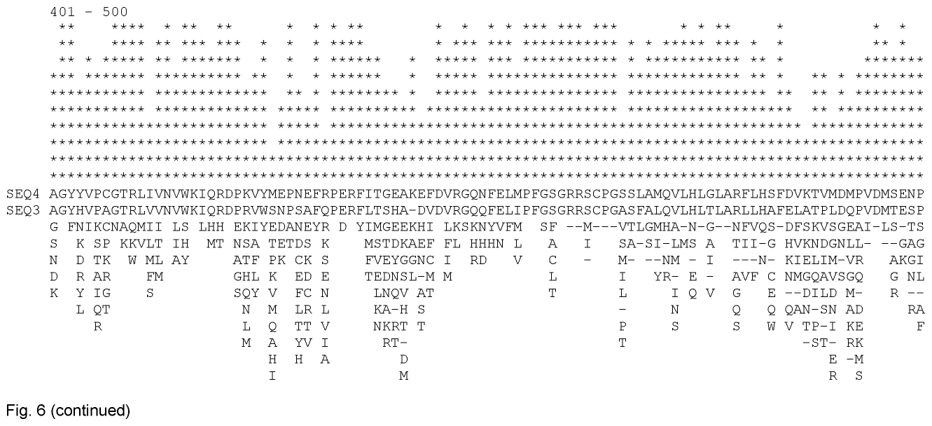

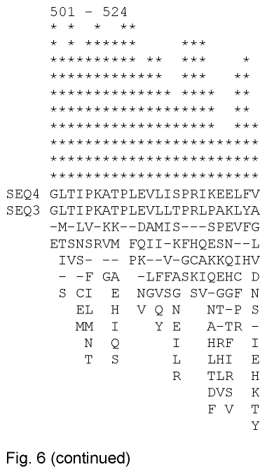

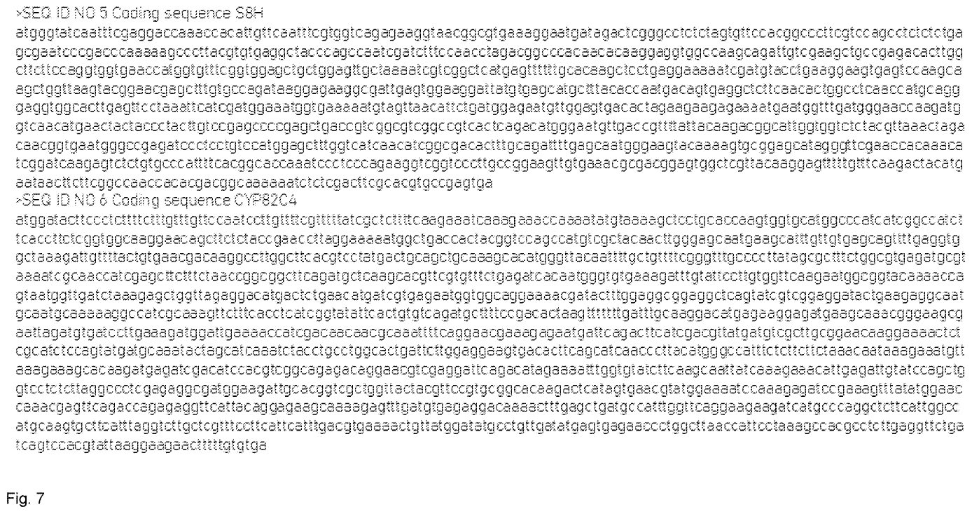

Even more preferably, the CYP82C4 enzyme sequence differs from the sequence according to SEQ ID NO. 3 only according to the amino acids given in FIG. 6 for each respective position. It has been found that these amino acids occur, at the respective positions, in homologous enzyme sequences. Thus, by limiting exchanges (substitutions, insertions or deletions) in for example a sequence according to one of the aforementioned Uniprot identifiers only to the sequences allowed according to this figure, the resulting mutant enzyme can have a restored or improved enzymatic activity.

Particularly preferred is an CYP82C4 enzyme having the sequence according to SEQ ID NO. 4, i.e. Uniprot identifier C82C4_ARATH. As shown in the examples below such enzyme has been found to be functional and highly active, thereby allowing to achieve the advantages of the present invention. Accordingly, it is preferred that the CYP82C4 enzyme sequence only differs from SEQ ID NO. 3 by one, more or all of the following mutations, in the numbering according to SEQ ID NO. 4: P3T, 16F, A7S, L10V, S11P, L12I, I13L, F14V, L15F, Y16V, K17F, V18I, L19A, G21F, -23K, -24S, S27P, S29Y, R30V, E31K, E34A, A36S, G51K, D52E, A61K, K65H, F70M, N71S, I72L, R73Q, R77N, R78E, W85F, E90D, I94V, T104M, V106A, Y115F, P125A, L136I, E145Q, V155I, D156T, I159V, R160K, E161D, N164S, V167F, Q168K, -170G, S172T, R173K, L176M, E178D, R181S, L186M, V190M, V191I, A203G, S204G, A205G, T206S, C207V, -208S, -209S, D210E, G212T, -213E, R216M, R217Q, Q219K, S223A, Q224K, V233T, L238F, F240T, W242S, W243F, L244F, A252E, K255Q, A257G, K258S, A262V, G266R, L268I, E270N, R274Q, V276K, S277F, G278S, K280T, A281K, G283N, Q285S, L292M, Q295A, E297Q, Q299K, N302H, F303L, D308N, T326S, G328S, P341K, E349D, L351I, L353I, K357R, E358D, Q360N, D362E, E363D, K367E, H404Y, A407C, V412I, R423K, W425Y, S426M, N427E, S429N, A430E, Q432R, L437I, S439G, H440E, -442K, D443E, V444F, Q450N, I454M, A466S, F468L, L470M, T476G, L480F, A483S, E485D, L486V, A487K, P489V, L490M, Q492M, T497S, S499N, L514I, T515S, L518I, P519K, A520E, K521E, Y523F, A524V. These mutations increase the identity between a candidate CYP82C4 enzyme sequence, e.g. according to one of the aforementioned Uniprot identifiers, and the most preferred sequence. Thus, such mutations provide a way to align a candidate enzyme sequence to the most preferred one and to improve its function, thereby achieving the advantages of the present invention.

Heterologous expression of the S8H and/or CYP82C4 enzyme can be achieved by introducing a heterologous expression cassette comprising a heterologous gene coding for the respective enzyme. Such cell would be a transgenic cell. Heterologous expression can also be achieved by mutating an existing coding frame coding for a protein of the IPR027443 or IPR005123 superfamily, respectively, to make its protein sequence conform to the detailed recommendations given herein. Such cell would be a recombinant cell. Where a pre-existing coding frame is mutated, it may be necessary to also provide a promoter and/or regulatory for expression of the mutated enzyme to prevent the coding frame from remaining silent. Preferably the plant cell is a transgenic plant cell, and/or the gene coding for the S8H and/or the CYP82C4 enzyme is operably linked to a heterologous promoter and/or terminator. Such cells are particularly easy to produce. An example of such plant cell production and of advantages thereof is described in the Examples section.

Expression of heterologous S8H and/or CYP82C4 enzymes preferably is achieved by stably transforming a plant cell with an exogenous nucleic acid comprising, for the respective S8H and/or CYP82C4 enzyme as described herein, an expression cassette wherein the gene coding for the respective enzyme is operably linked to a promoter, preferably a promoter is a constitutive promoter an inducible promoter, preferably a pathogen-inducible promoter, or a tissue-specific promoter, preferably a mesophyll-specific promoter or an epidermis specific-promoter and most preferably a stem and/or leaf specific promoter. After transformation of the plant cell, a plant is regenerated therefrom, expressing the exogenous nucleic acid. Such techniques are known to the skilled person and can be performed without undue burden, using common laboratory equipment and materials. Thus, it is an advantage of the present invention that it provides easily accessible materials and simple methods to increase or confer pathogen resistance, preferably fungal resistance, to crops, preferably soybean.

Correspondingly, the plant cell, and the respective plant part or plant comprising the cell, preferably comprises an exogenous nucleic acid, preferably integrated into the genome of the plant cell in a single full copy, wherein the exogenous nucleic acid comprises an expression cassette for the S8H gene and/or an expression cassette for the CYP82C4 gene as described herein. Preferred S8H and CYP82C4 enzymes are described herein. The skilled person can reverse translate the enzyme amino acid sequence into a correspondingly coding nucleic acid gene sequence. Preferably the gene sequence is adapted to the plant, more preferably the gene sequence conforms to the plant species' codon usage frequency.

The plant cell preferably comprises a metabolic pathway for the production of one or more coumarins, preferably esculetin, scopoletin and/or isoscopoletin. This advantageously avoids having to supply scopoletin to the plant cell or to the plant part comprising such plant cells or to the whole plant for conversion into fraxetin and, optionally, sideretin. As described above, genes for the production of scopoletin are described in WO2016124515 and WO2020120753. These documents are incorporated herein by reference. Thus, preferably the plant cell comprises and/or overexpresses an ferulate 6-hydroxylase (F6H1 enzyme) and optionally at least one or more additional protein(s) selected from the group consisting of a OMT3, 4-Coumarate-Coenzyme A ligase, CYP199A2, COSY, CCoAOMT, ABCG37 and UGT71C1. Such genes and combinations thereof allow for a particularly suitable production of coumarins, preferably of scopoletin, in plants to prevent, reduce or delay infections by phythopathogenic microorganisms, preferably of rust fungus infections in leguminous plants, most preferably in soybean. Thus, preferably the plant cell of the present invention comprises an expression cassette not only for the S8H and/or CYP82C4 gene of the present invention but also a further expression cassette for F6′H1 and preferably also one for OMT3.

Preferably the production of coumarins, most preferably at least of fraxetin and/or sideretin, is limited to those cells that are susceptible to pathogen interaction. Thus it is preferred that expression of the gene coding for the S8H and/or the CYP82C4 enzyme and/or of one or more genes, if present, of the metabolic pathway for production of one or more coumarins is/are directed in a way that expression occurs in root, stem and/or leaf cells, and preferably is reduced or repressed in fruit or seed cells. While coumarins have found applications in medicine, it is generally advantageous to reduce their content in such part of plant that are intended for human or non-pest-animal consumption, in particular fruit and seed cells. As described herein the present invention provides a way to combat infections by phythopathogenic microorganisms, preferably of fungi or oomycetes. Such infections generally occur via infections of stem or leaf cells. Thus, most preferably expression is regulated to occur not in root cells or only to a lesser degree in root cells compared to stem or leaf cells.

The invention also provides a plant or plant part comprising a plant cell of the present invention. Such plant or plant part benefits from the advantages imparted by the S8H and/or CYP82C4 enzyme(s), and in particular is less or not affected by increased scopoletin synthesis and preferably exhibits
coumarin accumulation on a surface of the plant or plant part, and/or
reduced, delayed or inhibited germination or growth of a phytopathogenic microorganism of a surface of the plant or plant part, and/or
increased resistance against infection by a phytopathogenic microorganism and/or increased resistance against parasitic plants.

The plant or part thereof according to the invention preferably exhibits, when compared to a corresponding wild type plant or part thereof,
new or increased production and/or accumulation of (a) fraxetin and optionally sideretin and/or of (b) a derivative thereof, and/or
modified, reduced or removed plant health reducing effects of the production of esculetin, scopoletin and/or isoscopoletin, and/or
reduced, delayed or inhibited germination or growth of a phytopathogenic microorganism on a surface of the plant or plant part, and/or
increased resistance against infection by a phytopathogenic microorganism and/or increased resistance against parasitic plants, wherein the phytopathogenic microorganism preferably is selected from any of
phylum Ascomycota, Basidiomycota or Oomycota, more preferably of
order Pleosporales, Heliotiales, Hypocreales or Pucciniales, more preferably of
genus Alternaria, Botrytis, Sclerotinia, Fusarium, or, most preferred, Phakopsora.

Preferred pathogenic microorganisms and the corresponding diseases are also indicated in tables 1 and 2 below.

TABLE 1
Diseases caused by biotrophic and/or heminecrotrophic
phytopathogenic fungi

| Disease | Pathogen |
|---|---|
| Leaf rust | Puccinia recondita |
| Yellow rust | P. striiformis |
| Powdery mildew | Erysiphe graminis/Blumeria graminis |
| Rust (common corn) | Puccinia sorghi |
| Rust (Southern corn) | Puccinia polysora |
| Tobacco leaf spot | Cercospora nicotianae |
| Rust (soybean) | Phakopsora pachyrhizi, P. meibomiae |
| Rust (tropical corn) | Physopella pallescens, P. zeae Angiopsora zeae |

TABLE 2
Diseases caused by necrotrophic and/or
hemibiotrophic fungi and oomycetes

| Disease | Pathogen |
|---|---|
| Plume blotch | Septoria (Stagonospora) nodorum |
| Leaf blotch | Septoria tritici |
| Ear fusarioses | Fusarium spp. |
| Late blight | Phytophthora infestans |
| Anthrocnose | Colletotrichum graminicola (teleomorph: |
| leaf blight, | Glomerella graminicola Politis); Glomerella |
| Anthracnose | tucumanensis (anamorph: Glomerella falcatum |
| stalk rot | Went) |
| Curvularia leaf | Curvularia clavata, C. eragrostidis, = C. maculans |
| spot | (teleomorph: Cochliobolus eragrostidis), Curvularia |
| | inaequalis, C. intermedia (teleomorph: Cochliobolus |
| | intermedius), Curvularia lunata (teleomorph: |
| | Cochliobolus lunatus), Curvularia pallescens |
| | (teleomorph: Cochliobolus pallescens), Curvularia |
| | senegalensis, C. tuberculata (teleomorph: |
| | Cochliobolus tuberculatus) |
| Didymella leaf | Didymella exitalis |
| spot | |
| Diplodia leaf | Stenocarpella macrospora = Diplodialeaf |
| spot or streak | macrospora |
| Brown stripe | Sclerophthora rayssiae var. zeae |
| downy mildew | |
| Crazy top | Sclerophthora macrospora = Sclerospora |
| downy mildew | macrospora |
| Green ear | Sclerospora graminicola |
| downy mildew | |
| (gramini-cola | |
| downy mildew) | |
| Leaf spots, | Alternaria alternata, Ascochyta maydis, A. tritici, A. |
| minor | zeicola, Bipolaris victoriae = Helminthosporium |
| | victoriae, (teleomorph: Cochliobolus victoriae), C. |
| | sativus (anamorph: Bipolaris sorokiniana = H. |
| | sorokinianum = H. sativum), Epicoccum nigrum, |
| | Exserohilum prolatum = Drechslera prolata |
| | (teleomorph: Setosphaeria prolata), Graphium |
| | penicillioides, Leptosphaeria maydis, Leptothyrium |
| | zeae, Ophiosphaerella herpotricha, (anamorph: |
| | Scolecosporiella sp.), Paraphaeosphaeria michotii, |
| | Phoma sp., Septoria zeae, S. zeicola, S. zeina |
| Northern corn | Setosphaeria turcica (anamorph: Exserohilum |
| leaf blight | turcicum = Helminthosporium turcicum) |
| (white blast, | |
| crown stalk | |
| rot, stripe) | |
| Northern corn | Cochliobolus carbonum (anamorph: Bipolaris |
| leaf spot | zeicola = Helminthosporium carbonum) |
| Helminthosporium | |
| ear rot (race 1) | |
| Phaeosphaeria leaf | Phaeosphaeria maydis = Sphaerulina maydis |
| spot | |
| Rostratum | Setosphaeria rostrata, (anamorph: xserohilum |
| leaf spot | rostratum = Helminthosporium rostratum) |
| (Helminthosporium | |
| leaf disease, ear | |
| and stalk rot) | |
| Java downy | Peronosclerospora maydis = Sclerospora maydis |
| mildew | |
| Philippine downy | Peronosclerospora philippinensis = Sclerospora |
| mildew | philippinensis |
| Sorghum downy | Peronosclerospora sorghi = Sclerospora sorghi |
| mildew | |
| Spontaneum | Peronosclerospora spontanea = Sclerospora |
| downy mildew | spontanea |
| Sugarcane downy | Peronosclerospora sacchari = Sclerospora sacchari |
| mildew | |
| Sclerotium ear rot | Sclerotium rolfsii Sacc. (teleomorph: Athelia rolfsii) |
| (southern blight) | |
| Seed rot-seedling | Bipolaris sorokiniana, B. zeicola = |
| blight | Helminthosporium carbonum, Diplodia maydis, |
| | Exserohilum pedicillatum, Exserohilum turcicum = |
| | Helminthosporium turcicum, Fusarium avenaceum, |
| | F. culmorum, F. moniliforme, Gibberella zeae |
| | (anamorph: F. graminearum), Macrophomina |
| | phaseolina, Penicillium spp., Phomopsis sp., |
| | Pythium spp., Rhizoctonia solani, R. zeae, |
| | Sclerotium rolfsii, Spicaria sp. |
| Selenophoma leaf | Selenophoma sp. |
| spot | |
| Yellow leaf blight | Ascochyta ischaemi, Phyllosticta maydis |
| | (teleomorph: Mycosphaerella zeae-maydis) |
| Zonate leaf spot | Gloeocercospora sorghi |

In particular, the invention provides leguminous crop plants and parts and cells thereof, most preferably soybean, comprising the S8H and/or CYP82C4 gene as described herein. Such plants, when producing a coumarin, most preferably scopoletin, are preferably protected against infection by a fungal pathogens of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae,
even more preferably of genus Rhizoctonia, Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis,
even more preferably of species Rhizoctonia alpina, Rhizoctonia bicornis, Rhizoctonia butinii, Rhizoctonia callae, Rhizoctonia carotae, Rhizoctonia endophytica, Rhizoctonia floccosa, Rhizoctonia fragariae, Rhizoctonia fraxini, Rhizoctonia fusispora, Rhizoctonia globularis, Rhizoctonia gossypii, Rhizoctonia muneratii, Rhizoctonia papayae, Rhizoctonia quercus, Rhizoctonia repens, Rhizoctonia rubi, Rhizoctonia silvestris, Rhizoctonia solani, Phakopsora ampelopsidis, Phakopsora apoda, Phakopsora argentinensis, Phakopsora cherimoliae, Phakopsora cingens, Phakopsora coca, Phakopsora crotonis, Phakopsora euvitis, Phakopsora gossypii, Phakopsora hornotina, Phakopsora jatrophicola, Phakopsora meibomiae, Phakopsora meliosmae, Phakopsora meliosmae-myrianthae, Phakopsora montana, Phakopsora muscadiniae, Phakopsora myrtacearum, Phakopsora nishidana, Phakopsora orientalis, Phakopsora pachyrhizi, Phakopsora phyllanthi, Phakopsora tecta, Phakopsora uva, Phakopsora vitis, Phakopsora ziziphi-vulgaris,
Puccinia abrupta, Puccinia acetosae, Puccinia achnatheri-sibirici, Puccinia acroptili, Puccinia actaeae-agropyri, Puccinia actaeae-elymi, Puccinia antirrhini, Puccinia argentata, Puccinia arrhenatheri, Puccinia arrhenathericola, Puccinia artemisiae-keiskeanae, Puccinia arthrocnemi, Puccinia asteris, Puccinia atra, Puccinia aucta, Puccinia ballotiflora, Puccinia bartholomaei, Puccinia bistortae, Puccinia cacabata, Puccinia calcitrapae, Puccinia calthae, Puccinia calthicola, Puccinia calystegiae-soldanellae, Puccinia canaliculata, Puccinia caricis-montanae, Puccinia caricis-stipatae, Puccinia carthami, Puccinia cerinthes-agropyrina, Puccinia cesatii, Puccinia chrysanthemi, Puccinia circumdata, Puccinia clavata, Puccinia coleataeniae, Puccinia coronata, Puccinia coronati-agrostidis, Puccinia coronati-brevispora, Puccinia coronati-calamagrostidis, Puccinia coronati-hordei, Puccinia coronati-japonica, Puccinia coronati-longispora, Puccinia crotonopsidis, Puccinia cynodontis, Puccinia dactylidina, Puccinia dietelii, Puccinia digitata, Puccinia distincta, Puccinia duthiae, Puccinia emaculata, Puccinia erianthi, Puccinia eupatorii-columbiani, Puccinia flavenscentis, Puccinia gastrolobii, Puccinia geitonoplesii, Puccinia gigantea, Puccinia glechomatis, Puccinia helianthi, Puccinia heterogenea, Puccinia heterospora, Puccinia hydrocotyles, Puccinia hysterium, Puccinia impatientis, Puccinia impedita, Puccinia imposita, Puccinia infra-aequatorialis, Puccinia insolita, Puccinia justiciae, Puccinia klugkistiana, Puccinia knersvlaktensis, Puccinia lantanae, Puccinia lateritia, Puccinia latimamma, Puccinia liberta, Puccinia littoralis, Puccinia lobata, Puccinia lophatheri, Puccinia loranthicola, Puccinia menthae, Puccinia mesembryanthemi, Puccinia meyeri-albertii, Puccinia miscanthi, Puccinia miscanthidii, Puccinia mixta, Puccinia montanensis, Puccinia morata, Puccinia morthieri, Puccinia nitida, Puccinia oenanthes-stoloniferae, Puccinia operta, Puccinia otzeniani, Puccinia patriniae, Puccinia pentstemonis, Puccinia persistens, Puccinia phyllostachydis, Puccinia pittieriana, Puccinia platyspora, Puccinia pritzeliana, Puccinia prostii, Puccinia pseudodigitata, Puccinia pseudostriiformis, Puccinia psychotriae, Puccinia punctata, Puccinia punctiformis, Puccinia recondita, Puccinia rhei-undulati, Puccinia rupestris, Puccinia senecionis-acutiformis, Puccinia septentrionalis, Puccinia setariae, Puccinia silvatica, Puccinia stipina, Puccinia stobaeae, Puccinia striiformis, Puccinia striiformoides, Puccinia stylidii, Puccinia substriata, Puccinia suzutake, Puccinia taeniatheri, Puccinia tageticola, Puccinia tanaceti, Puccinia tatarinovii, Puccinia tetragoniae, Puccinia thaliae, Puccinia thlaspeos, Puccinia tillandsiae, Puccinia tiritea, Puccinia tokyensis, Puccinia trebouxi, Puccinia triticina, Puccinia tubulosa, Puccinia tulipae, Puccinia tumidipes, Puccinia turgida, Puccinia urticae-acutae, Puccinia urticae-acutiformis, Puccinia urticae-caricis, Puccinia urticae-hirtae, Puccinia urticae-inflatae, Puccinia urticata, Puccinia vaginatae, Puccinia virgata, Puccinia xanthii, Puccinia xanthosiae, Puccinia zoysiae, more preferably of species Phakopsora pachyrhizi, Puccinia graminis, Puccinia striiformis, Puccinia hordei or Puccinia recondita, more preferably of genus Phakopsora and most preferably Phakopsora pachyrhizi. As indicated above, fungi of these taxa are responsible for grave losses of crop yield. This applies in particular to rust fungi of genus Phakopsora. It is thus an advantage of the present invention that the method allows to reduce fungicide treatments against Phakopsora pachyrhizi. Furthermore, conversion of scopoletin into fraxetin by the S8H enzyme and optionally further conversion of fraxetin to sideretin by the CYP82C4 enzyme reduces plant health adverse effects of heterologous coumarin synthesis.

Preferably the plant, plant part or plant cell is soybean, beans, pea, clover, kudzu, lucerne, lentils, lupins, vetches, groundnut, rice, wheat, barley, Arabidopsis, lentil, banana, canola, cotton, potato, maize, sugar cane, alfalfa, sugar beet, sunflower, rapeseed, sorghum, rice, cabbage, tomato, peppers, sugar cane and tobacco.

It is preferred according to the invention that the plant is a crop plant, preferably a dikotyledon, more preferably not of sub-family Solanoidae, more preferably not of family Solanaceae, more preferably a plant of order Fabales, more preferably a plant of family Fabaceae, more preferably a plant of tribus Phaseoleae, more preferably of genus Amphicarpaea, Cajanus, Canavalia, Dioclea, Erythrina, Glycine, Arachis, Lathyrus, Lens, Pisum, Vicia, Vigna, Phaseolus or Psophocarpus, even more preferably of species Amphicarpaea bracteata, Cajanus cajan, Canavalia brasiliensis, Canavalia ensiformis, Canavalia gladiata, Dioclea grandiflora, Erythrina latissima, Phaseolus acutifolius, Phaseolus lunatus, Phaseolus maculatus, Psophocarpus tetragonolobus, Vigna angularis, Vigna mungo, Vigna unguiculata, Glycine albicans, Glycine aphyonota, Glycine arenaria, Glycine argyrea, Glycine canescens, Glycine clandestina, Glycine curvata, Glycine cyrtoloba, Glycine dolichocarpa, Glycine falcata, Glycine gracei, Glycine hirticaulis, Glycine lactovirens, Glycine latifolia, Glycine latrobeana, Glycine microphylla, Glycine peratosa, Glycine pindanica, Glycine pullenii, Glycine rubiginosa, Glycine stenophita, Glycine syndetika, Glycine tabacina, Glycine tomentella, Glycine gracilis, Glycine max, Glycine max×Glycine soja, Glycine soja, more preferably of species Glycine gracilis, Glycine max, Glycine max×Glycine soja, Glycine soja, most preferably of species Glycine max. As shown herein particularly good plant health improvements are obtained for soybean.

The crop may comprise, in addition to the heterologous expression cassette, one or more furthe heterologous elements. For example, transgenic soybean events comprising herbicide tolerance genes are for example, but not excluding others, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHT0H2, W62, W98, FG72 and CV127; transgenic soybean events comprising genes for insecticidal proteins are for example, but not excluding others, MON87701, MON87751 and DAS-81419. Cultivated plants comprising a modified oil content have been created by using the transgenes: gm-fad2-1, Pj.D6D, Nc.Fad3, fad2-1A and fatb1-A. Examples of soybean events comprising at least one of these genes are: 260-05, MON87705 and MON87769. Plants comprising such singular or stacked traits as well as the genes and events providing these traits are well known in the art. For example, detailed information as to the mutagenized or integrated genes and the respective events are available from websites of the organizations International Service for the Acquisition of Agrl. biotech Applications (ISAAA) (http://www.isaaa.org/gmapprovaldatabase) and the Center for Environmental Risk Assessment (CERA) (http://cera-qmc.org/GMCropDatabase). Further information on specific events and methods to detect them can be found for soybean events H7-1, MON89788, A2704-12, A5547-127, DP305423, DP356043, MON87701, MON87769, CV127, MON87705, DAS68416-4, MON87708, MON87712, SYHT0H2, DAS81419, DAS81419×DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

It is a particular advantage of the present invention that the benefits of the present invention do not require homozygous plants producing the S8H and CYP82C4 enzymes but is also applicable for hemizygous or heterozygous plants. The invention thus also provides plant progeny obtained by breeding a plant of the present invention, wherein the progeny comprises the heterologous S8H and/or CYP82C4 gene.

As described herein it is an advantage that the plants of the present invention, comprising plant cells of the present invention, show improved plant health compared to coumarin- and particularly scopoletin-producing control plants. Furthermore such plants are less prone to infections by microbial pathogens. In particular soybeans show an improved resistance against soybean rust. Thus, such plants advantageously require less intensive pesticide treatments, in particular soybean plants of the invention require less treatments against soybean rust. The achievable reduced exposition to pesticides improves consumer appreciation of products of such plants.

Preferably, the production and/or accumulation of coumarins, more preferably of fraxetin and/or, more preferably, of sideretin, is increased in the stem and/or a leaf compared to a wild type plant. As described herein, stems and leaves are most susceptible to infections by plant pathogens; notably soybean leaves are affected by soybean rust infections. Thus, it is an advantage of the present invention that the plant health improving coumarins, preferably fraxetin and most preferably sideretin, can be particularly strongly present and focused in those plant parts most in need thereof. This also limits plant health adverse effects of coumarin production in those plant parts with a lesser demand for sideretin, fraxetin or other coumarins, for example fruit, seed and root.

The invention thus also provides a non-propagative plant part or material of a plant or plant part, preferably a fermentation product, oil, meal, press cake, pomace, chaff, straw or compost.

And the invention provides a product of a plant, plant part or plant cell of the present invention, wherein the product is obtainable or obtained by
i) collecting a material of said plant, plant part or plant cell, preferably a harvestable plant part and most preferably a plant seed, and
ii) disrupting the collected material, preferably to obtain a fermentation product, oil, meal, press cake, pomace, chaff, straw or compost.

Such products also benefit from the reduced need for pesticide treatments and advantageously comprise a reduced content of pesticide degradation products and/or plant pathogen metabolic products.

The invention also provides a method for improving plant health, comprising the step of conferring or increasing the production and/or accumulation of (a) fraxetin and optionally sideretin and/or of (b) a derivative thereof, in a plant cell, plant part or whole plant in comparison to a respective wild type plant, wild type plant part, or wild type plant cell.

As described above, plant health improvement preferably manifests itself in
modifying, reducing or removing plant health reducing effects of the production of esculetin, scopoletin and/or isoscopoletin
reducing, delaying or inhibiting germination or growth of a phytopathogenic microorganism on a surface of the plant or plant part, and/or
increasing resistance against infection by a phytopathogenic microorganism and/or increasing resistance against parasitic plants, wherein the phytopathogenic microorganism preferably is selected from any of
phylum Ascomycota, Basidiomycota or Oomycota, more preferably of
order Pleosporales, Heliotiales, Hypocreales or Pucciniales, more preferably of
genus Alternaria, Botrytis, Sclerotinia, Fusarium, or, most preferred, Phakopsora.

As described above, the plant cell, plant part or plant of the plant health improving method of the invention preferably is a soybean plant cell, plant part or plant. It is a particular advantage of the present invention that the synthesis of fraxetin, and preferably of sideretin, can be increased in non-root plant parts and non-root plant cells. Thus, the present invention advantageously provides an improved protection against plant pathogens, in particular against rust fungi, preferably of genus Phakopsora in view of soybean plants. Thus, it is an advantage that the present invention provides a method for conferring or increasing fungal resistance in a soybean plant, a plant part, or a plant cell, wherein the method comprises the step of increasing the production and/or accumulation of fraxetin and/or sideretin in the plant, plant part, or plant cell in comparison to a respective wild type plant, wild type plant part, or wild type plant cell.

Preferably, the accumulation or production of fraxetin and/or, preferably, sideretin is increased in the stem and/or, more preferably, in leaves of the plant. This way, the pesticidal effect of fraxetin and/or sideretin is highest in those plant parts which have the greatest need for protection against pathogens, in particular against rust pathogens, preferably against infections by Phakopsora pachyrhizi. By selectively increasing the coumarin production and/or accumulating in these organs that have greatest need thereof, plant health averse effects of coumarin production can be further reduced.

The production or content of fraxetin and/or, more preferably, of sideretin, or of a derivative thereof, preferably is achieved by increasing the expression and/or activity of an S8H and/or optionally an CYP82C4 enzyme in the plant, plant part or plant cell, wherein the plant, plant part or plant cell comprises a metabolic pathway for the production of esculetin, scopoletin and/or isoscopoletin. Preferred S8H and CYP82C4 enzymes are described above.

The invention also provides an automated plant selection method, comprising the steps of
i) obtaining, for each seed of a plurality of seeds, a sample comprising genetic material of a tissue body representative for said seed,
ii) determining the presence of a S8H and/or CYP82C4 gene as described herein in the genetic material, and optionally the presence of one or more genes of a metabolic pathway for production of one or more coumarins,
iii) selecting those seed where the determination in step ii) gave a positive result.

It is a particular advantage that the invention provides, as described above, a heritable trait whose presence can be easily ascertained in offspring plants even before germination of the plants and exposition to phythopathogenic microorganisms, thereby allowing for a particularly fast breeding process. Such processes can advantageously further be sped up by automatically analysing tissue material representative of an individual seed of a plurality of seeds, e.g. DNA of testa or of seed cells. Thus, seeds that do not contain a functional S8H and/or CYP82C4 gene according to the invention can automatically be discarded, thereby reducing the content of non-trait harbouring material in offspring generated by breeding.

In view of the aforementioned effects and advantages, the invention also teaches, preferably in relation to soybean plants, plant parts and plant cells, the use of an S8H and/or CYP82C4 enzyme or of a nucleic acid comprising an expression cassette comprising an S8H gene and/or a CYP82C4 gene for any of:
i) conferring or increasing the production and/or accumulation of (a) fraxetin and optionally sideretin and/or of (b) a derivative thereof, in a plant cell, plant part or whole plant,
ii) improving plant health, preferably
   modifying, reducing or removing plant health reducing effects of the production of esculetin, scopoletin and/or isoscopoletin
   reducing, delaying or inhibiting germination or growth of a phytopathogenic microorganism on a surface of the plant or plant part, and/or
   increasing resistance against infection by a phytopathogenic microorganism and/or increasing resistance against parasitic plants, wherein the phytopathogenic microorganism preferably is selected from any of
   phylum Ascomycota, Basidiomycota or Oomycota, more preferably of
   order Pleosporales, Heliotiales, Hypocreales or Pucciniales, more preferably of
   genus Alternaria, Botrytis, Sclerotinia, Fusarium, or, most preferred, Phakopsora.

Selected aspects of the invention are hereinafter described in more detail by non-limiting examples.

### EXAMPLES

### Example 1: Growth of N. benthamiana

N. benthamiana lines were first sown and grown for two weeks under long-day conditions (16 h light and 8 h darkness at 22° C., 70% relative humidity and 85 μmol m-2 s-1 light intensity). After two weeks plants were repotted and grown in the green house until further use.

### Example 2: Cloning for Transient Transformation Assays

Coding sequences (CDS) of the biosynthesis genes AtS8H (AT3G12900.1) and AtCYP82C4 (AT4G31940.1) for cloning DNA constructs were amplified from Arabidopsis cDNA. For the generation of overexpression constructs, the CDS was cloned under a 35S-promoter into a modified pK7GWIWG2-7F2,1 DNA vector (The VIB-UGent PSB Plasmid Repository). For this, a Gibson assembly was performed as described in the literature using modified pK7GWIWG2-7F2,1 vector linearised with XhoI and KpnI (Thermo Fisher) (Gibson D G, Young L, Chuang R-Y, Venter J C, Hutchison C a, et al. Enzymatic assembly of DNA molecules up to several hundred kilobases. Nat Methods 2009; 6:343-5).

### Example 3: Transient Transformation of N. benthamiana

For transient transformation of N. benthamiana 4-6-week-old plants were used according to a modified protocol from Popescu et al. (Popescu S C, Popescu G V., Bachan S, Zhang Z, Seay M, et al. Differential binding of calmodulin-related proteins to their targets revealed through high-density Arabidopsis protein microarrays. Proc Natl Acad Sci USA 2007; 104:4730-4735). Agrobacteria (AGL01) containing the DNA construct of interest were grown for 14-16 h in YEB medium with the respective antibiotics, shaking at 220 rpm and 28° C. Cells were then harvested by centrifugation at 4000 g for 15 min and resuspended in infiltration medium (10 mM MES, 10 mM MgCl2, 150 μM Acetosyringone, pH 5.6) and set to an OD600 of 0.5. After incubating for 2 h at RT, the suspension was mixed with an equal volume of Agrobacteria containing the p19 silencing suppressor gene from tomato bushy stunt virus (TBSV). N. benthamiana leaves were then syringe-infiltrated with the 1:1 mixtures. Plants were placed under long-day conditions for three days, before leaves were harvested for further analysis.

### Example 4: Coumarin Extraction from Plant Tissue

For the extraction of coumarins, plant material was frozen, ground and 200 mg extracted with 1 ml 90% methanol for 16 h on a spinning wheel, to ensure constant mixing. Subsequently plant debris was pelleted by centrifugation and the supernatant transferred t a fresh reaction tube and evaporated. The resulting pellet was resuspended in 300 μl methanol and resuspended by vortexing. Samples were then stored at −20° C. until being used for HPLC analysis

### Example 5: High Performance Liquid Chromatography (HPLC)

A 10 μl aliquot of the extract was separated on a C18 reverse phase column (NUCLEODUR 100-5 C18 ec, Macherey-Nagel) heated to 40° C. The solvent gradient used for separation of coumarins can be taken from Table 3. Fluorescent coumarins were detected in the fluorescence detector (RF-20A; Shimadzu Corp.) at 365 nm excitation and 470 nm emission. Non-fluorescent coumarins, such as fraxetin and sideretin, were detected in the photodiode array at 342 nm absorption (SPD-M40, Shimadzu Corp.) and an absorption spectrum (230-500 nm) plotted. A complete setup of all HPLC components can be viewed in Table 4.

TABLE 3
Solvent gradients for HPLC analysis. Solvent gradient
for separation of coumarins. ddH2O and acetonitril
for separation of coumarins contained 1.5% (v/v)
acetic acid. The flow rate was set to 0.8 ml/min.

| time in min | ddH2O (%) | Actonitril (%) |
|---|---|---|
| 0 | 90 | 10 |
| 10 | 80 | 20 |
| 19 | 66 | 34 |
| 20 | 10 | 90 |
| 23 | 10 | 90 |
| 24 | 90 | 10 |
| 27 | 90 | 10 |

TABLE 4
List of HPLC components used. All components
used here were from Shimadzu Corp.

| HPLC component | Product number |
|---|---|
| Degasser unit | DGU-403 |
| Solvent delivery module | LC-40D |
| Auto sampler | SIL-40 |
| Column oven | CTO-40S |
| Fluorescence detector | RF-20A |
| Photo diode array detector | SPD-M40 |

### Example 6: QPCR Analysis of Expression of Biosynthesis Genes in Transgenic N. benthamiana

For extraction of RNA from N. benthamiana plant material the Trizol-based method introduced by Chomczynski and Sacchi was used (Chomczynski P, Sacchi N. Single-step method of RNA isolation by acid guanidinium thiocyanate-phenol-chloroform extraction. Anal Biochem 1987; 162:156-159).

For synthesis of cDNA RevertAid™ M-MuLV Reverse Transcriptase (Thermo Fisher) was used as described by the manufacturer. Random primers were used during synthesis of cDNA for qRT-PCR analysis. This cDNA was subsequently diluted 1:10 (v/v) with ddH2O, before being used as template for qRT-PCR to quantify the transcript abundance of genes of interest. Measurement of abundance was conducted in a CFX-384 REAL-TIME system (BIO-RAD) using iTaq™ Universal SYBRR Green Supermix (BIO-RAD) following manufacturer's instructions. Expression of genes was normalised to NbActin (N. benthamiana) or GmUBQ3 (soybean).

### Example 7: Cloning for Stable Soybean Transformation

The DNA sequences of S8H (SEQ ID NO. 5), CYP82C4 (SEQ ID NO. 6), F6′H1 (WO2016124515) and CoSy (Vanholme, R., Sundin, L., Seetso, K. C. et al. COSY catalyses trans-cis isomerization and lactonization in the biosynthesis of coumarins. Nat. Plants 5, 1066-1075 (2019). https://doi.org/10.1038/s41477-019-0510-0) and all mentioned gene control element (promoters, terminators) were generated by DNA synthesis (Geneart, Regensburg, Germany).

To assemble the different expression cassettes the Gateway System® was used (Invitrogen, Life Technologies, Carlsbad, California, USA).

The pSuper::S8H::StCATHD-pA expression cassette was cloned into a pENTRY vector containing att4:1 recombination sites as a PacI/FseI fragment. The Super promoter was cloned as a PacI/Asci fragment. The S8H CDS was cloned as an AscI/SbfI fragment. The StCATHD-pA terminator was cloned as an SbfI/FseI fragment.

The PubiPc::CYP82C4::NOS expression cassette was also cloned as a PacI/FseI entity into an intermediate cloning vector. The PubiPc promoter was cloned as a PacI/AscI fragment. The CYP82C4 CDS was cloned as an AscI/SbfI fragment. The NOS terminator was cloned as an SbfI/FseI fragment. The complete PubiPc::CYP82C4::NOS expression cassette was excised from the cloning vector using SwaI (blunt end)/NotI (sticky end) and ligated into PmeI (blunt end)/NotI (sticky end) cut pENTRY (att4:1) vector containing the above mentioned Super::S8H::StCATHD-pA expression cassette to generate one double expression cassette ENTRY (att4:1) vector containing both S8H and CYP82C4: expression cassettes.

The PubiPc:AtF6H1::AgroOCS expression cassette was cloned into a pENTRY vector containing the att2:3 recombination sites as a PacI/FseI fragment. The PubiPc promoter was cloned as a PacI/AscI fragment. The AtF6H1 CDS was cloned as an AscI/SbfI fragment. The AgroOCS_192bp terminator was cloned as an SbfI/FseI fragment.

The Super::CoSy::OCS3 expression cassette was cloned into a pENTRY vector containing the att1:2 recombination sites as a PacI/FseI fragment. The Super promoter was cloned as a PacI/AscI fragment. The CoSy CDS was cloned as an AscI/SbfI fragment. The OCS3 terminator was cloned as an SbfI/FseI fragment.

To generate the binary plant transformation vector containing the above described Super::S8H::StCATHD-pA, PubiPc::CYP82C4::NOS, PubiPc:AtF6H1::AgroOCS and Super::CoSy::OCS3 expression cassettes, a LR reaction (Gateway system, Invitrogen, Life Technologies, Carlsbad, California, USA) was performed according to manufacturer's protocol. As target a binary pDEST vector was used which is composed of: (1) a spectinomycin/streptomycin resistance cassette for bacterial selection (2) a pVS1 origin for replication in Agrobacteria (3) a ColE1 origin of replication for stable maintenance in E. coli and (4) between the right and left border an AHAS selection under control of an AtAHASL-promoter. The recombination reaction was trans-formed into E. coli (DH5alpha), mini-prepped and screened by specific restriction digestions. A positive clone from each vector construct was sequenced and submitted to soybean transformation.

### Example 8: Soybean Transformation

The expression vector constructs (see example 7) is transformed into soybean.

### 8.1 Sterilization and Germination of Soybean Seeds

Virtually any seed of any soybean variety can be employed in the method of the invention. A variety of soybean cultivar (including Jack, Williams 82, Jake, Stoddard, CD215 and Resnik) is appropriate for soybean transformation. Soybean seeds are sterilized in a chamber with a chlorine gas produced by adding 3.5 ml 12N HCl drop wise into 100 ml bleach (5.25% sodium hypochlorite) in a desiccator with a tightly fitting lid. After 24 to 48 hours in the chamber, seeds are removed and approximately 18 to 20 seeds are plated on solid GM medium with or without 5 μM 6-benzyl-aminopurine (BAP) in 100 mm Petri dishes. Seedlings without BAP are more elongated and roots develop especially secondary and lateral root formation. BAP strengthens the seedling by forming a shorter and stockier seedling.

Seven-day-old seedlings grown in the light (>100 μEinstein/m2s) at 25 degree C. are used for explant material for the three-explant types. At this time, the seed coat was split, and the epicotyl with the unifoliate leaves are grown to, at minimum, the length of the cotyledons. The epicotyl should be at least 0.5 cm to avoid the cotyledonary-node tissue (since soybean cultivars and seed lots may vary in the developmental time a description of the germination stage is more accurate than a specific germination time).

For inoculation of entire seedlings, see Method A (example 8.3, and 8.3.2) or leaf explants see Method B (example 8.3.3).

For method C (see example 8.3.4), the hypocotyl and one and a half or part of both cotyledons are removed from each seedling. The seedlings are then placed on propagation media for 2 to 4 weeks. The seedlings produce several branched shoots to obtain explants from. The majority of the explants originated from the plantlet growing from the apical bud. These explants are preferably used as target tissue.

### 8.2—Growth and Preparation of Agrobacterium Culture

Agrobacterium cultures are prepared by streaking Agrobacterium (e.g., A. tumefaciens or A. rhizogenes) carrying the desired binary vector (e.g. H. Klee. R. Horsch and S. Rogers 1987 Agrobacterium-Mediated Plant Transformation and its further Applications to Plant Biology; Annual Review of Plant Physiology Vol. 38: 467-486) onto solid YEP growth medium YEP media: 10 g yeast extract. 10 g Bacto Peptone. 5 g NaCl. Adjust pH to 7.0, and bring final volume to 1 liter with H2O, for YEP agar plates add 20 g Agar, autoclave) and incubating at 25. degree C. until colonies appeared (about 2 days). Depending on the selectable marker genes present on the Ti or Ri plasmid, the binary vector, and the bacterial chromosomes, different selection compounds are to be used for A. tumefaciens and A. rhizogenes selection in the YEP solid and liquid media. Various Agrobacterium strains can be used for the transformation method.

After approximately two days, a single colony (with a sterile toothpick) is picked and 50 ml of liquid YEP is inoculated with antibiotics and shaken at 175 rpm (25° C.) until an OD600 between 0.8-1.0 is reached (approximately 2 d). Working glycerol stocks (15%) for transformation are prepared and one-ml of Agrobacterium stock aliquoted into 1.5 ml Eppendorf tubes then stored at −80° C.

The day before explant inoculation, 200 ml of YEP are inoculated with 5 μl to 3 ml of working Agrobacterium stock in a 500 ml Erlenmeyer flask. The flask is shaken overnight at 25° C. until the OD600 is between 0.8 and 1.0. Before preparing the soybean explants, the Agrobacteria ARE pelleted by centrifugation for 10 min at 5,500×g at 20° C. The pellet is suspended in liquid CCM to the desired density (OD600 0.5-0.8) and placed at room temperature at least 30 min before use.

### 8.3—Explant Preparation and Co-Cultivation (Inoculation)

### 8.3.1 Method A:

Explant Preparation on the Day of Transformation. Seedlings at this time had elongated epicotyls from at least 0.5 cm but generally between 0.5 and 2 cm. Elongated epicotyls up to 4 cm in length are successfully employed. Explants are then prepared with: i) with or without some roots, ii) with a partial, one or both cotyledons, all preformed leaves are removed including apical meristem, and the node located at the first set of leaves is injured with several cuts using a sharp scalpel.

This cutting at the node not only induces Agrobacterium infection but also distributes the axillary meristem cells and damaged pre-formed shoots. After wounding and preparation, the explants are set aside in a Petri dish and subsequently co-cultivated with the liquid CCM/Agrobacterium mixture for 30 minutes. The explants are then removed from the liquid medium and plated on top of a sterile filter paper on 15×100 mm Petri plates with solid co-cultivation medium. The wounded target tissues are placed such that they are in direct contact with the medium.

### 8.3.2 Modified Method A: Epicotyl Explant Preparation

Soybean epicotyl segments prepared from 4 to 8 d old seedlings are used as explants for regeneration and transformation. Seeds of soybean are germinated in 1/10 MS salts or a similar composition medium with or without cytokinins for 4 to 8 d. Epicotyl explants are prepared by removing the cotyledonary node and stem node from the stem section. The epicotyl is cut into 2 to 5 segments. Especially preferred are segments attached to the primary or higher node comprising axillary meristematic tissue.

The explants are used for Agrobacterium infection. Agrobacterium AGL1 harboring a plasmid with the gene of interest (GOI) and the AHAS, bar or dsdA selectable marker gene is cultured in LB medium with appropriate antibiotics overnight, harvested and suspended in a inoculation medium with acetosyringone. Freshly prepared epicotyl segments are soaked in the Agrobacterium suspension for 30 to 60 min and then the explants were blotted dry on sterile filter papers. The inoculated explants are then cultured on a co-culture medium with L-cysteine and TTD and other chemicals such as acetosyringone for increasing T-DNA delivery for 2 to 4 d. The infected epicotyl explants are then placed on a shoot induction medium with selection agents such as imazapyr (for AHAS gene), glufosinate (for bar gene), or D-serine (for dsdA gene). The regenerated shoots are subcultured on elongation medium with the selective agent.

For regeneration of transgenic plants, the segments are then cultured on a medium with cytokinins such as BAP, TDZ and/or Kinetin for shoot induction. After 4 to 8 weeks, the cultured tissues are transferred to a medium with lower concentration of cytokinin for shoot elongation. Elongated shoots are transferred to a medium with auxin for rooting and plant development. Multiple shoots are regenerated. Many stable transformed sectors showing strong cDNA expression are recovered. Soybean plants are regenerated from epicotyl explants. Efficient T-DNA delivery and stable transformed sectors are demonstrated.

### 8.3.3 Method B: Leaf Explants

For the preparation of the leaf explant the cotyledon is removed from the hypocotyl. The cotyledons are separated from one another and the epicotyl is removed. The primary leaves, which consist of the lamina, the petiole, and the stipules, are removed from the epicotyl by carefully cutting at the base of the stipules such that the axillary meristems are included on the explant. To wound the explant as well as to stimulate de novo shoot formation, any pre-formed shoots are removed and the area between the stipules was cut with a sharp scalpel 3 to 5 times. The explants are either completely immersed or the wounded petiole end dipped into the Agrobacterium suspension immediately after explant preparation. After inoculation, the explants are blotted onto sterile filter paper to remove excess Agrobacterium culture and place explants with the wounded side in contact with a round 7 cm Whatman paper overlaying the solid CCM medium (see above). This filter paper prevents A. tumefaciens overgrowth on the soybean-explants. Wrap five plates with Parafilm™ “M” (American National Can, Chicago, Ill., USA) and incubate for three to five days in the dark or light at 25° C.

### 8.3.4 Method C: Propagated Axillary Meristem

For the preparation of the propagated axillary meristem explant propagated 3-4 week-old plantlets are used. Axillary meristem explants can be pre-pared from the first to the fourth node. An average of three to four explants could be obtained from each seedling. The explants are prepared from plantlets by cutting 0.5 to 1.0 cm below the axillary node on the internode and removing the petiole and leaf from the explant. The tip where the axillary meristems lie is cut with a scalpel to induce de novo shoot growth and allow access of target cells to the Agrobacterium. Therefore, a 0.5 cm explant included the stem and a bud. Once cut, the explants are immediately placed in the Agrobacterium suspension for 20 to 30 minutes. After inoculation, the explants are blotted onto sterile filter paper to remove excess Agrobacterium culture then placed almost completely immersed in solid CCM or on top of a round 7 cm filter paper overlaying the solid CCM, depending on the Agrobacterium strain. This filter paper prevents Agrobacterium overgrowth on the soybean explants. Plates are wrapped with Parafilm™ “M” (American National Can, Chicago, Ill., USA) and incubated for two to three days in the dark at 25° C.

### 8.4—Shoot Induction

After 3 to 5 days co-cultivation in the dark at 25° C., the explants are rinsed in liquid SIM medium (to remove excess Agrobacterium) (SIM, see Olhoft et al 2007 A novel Agrobacterium rhizogenes-mediated transformation method of soybean using primary-node explants from seedlings In Vitro Cell. Dev. Biol.—Plant (2007) 43:536-549; to remove excess Agrobacterium) or Modwash medium (1×B5 major salts, 1×B5 minor salts, 1×MSIII iron, 3% Sucrose, 1×B5 vitamins, 30 mM MES, 350 mg/L Timentin pH 5.6, WO 2005/121345) and blotted dry on sterile filter paper (to prevent damage especially on the lamina) before placing on the solid SIM medium. The approximately 5 explants (Method A) or 10 to 20 (Methods B and C) explants are placed such that the target tissue was in direct contact with the medium. During the first 2 weeks, the explants could be cultured with or without selective medium. Preferably, explants are transferred onto SIM without selection for one week.

For leaf explants (Method B), the explant should be placed into the medium such that it is perpendicular to the surface of the medium with the petiole imbedded into the medium and the lamina out of the medium.

For propagated axillary meristem (Method C), the explant is placed into the medium such that it is parallel to the surface of the medium (basipetal) with the explant partially embedded into the medium.

Wrap plates with Scotch 394 venting tape (3M, St. Paul, Minn., USA) are placed in a growth chamber for two weeks with a temperature averaging 25.degree. C. under 18 h light/6 h dark cycle at 70-100 μE/m2s. The explants remain on the SIM medium with or without selection until de novo shoot growth occurred at the target area (e.g., axillary meristems at the first node above the epicotyl). Transfers to fresh medium can occur during this time. Explants are transferred from the SIM with or without selection to SIM with selection after about one week. At this time, there is considerable de novo shoot development at the base of the petiole of the leaf explants in a variety of SIM (Method B), at the primary node for seedling explants (Method A), and at the axillary nodes of propagated explants (Method C).

Preferably, all shoots formed before transformation are removed up to 2 weeks after co-cultivation to stimulate new growth from the meristems. This helped to reduce chimerism in the primary transformant and increase amplification of transgenic meristematic cells. During this time the explant may or may not be cut into smaller pieces (i.e. detaching the node from the explant by cutting the epicotyl).

### 8.5—Shoot Elongation

After 2 to 4 weeks (or until a mass of shoots is formed) on SIM medium (preferably with selection), the explants are transferred to SEM medium (shoot elongation medium, see Olhoft et al 2007 A novel Agrobacterium rhizogenes-mediated transformation method of soybean using primary-node explants from seedlings. In Vitro Cell. Dev. Biol.—Plant (2007) 43:536-549) that stimulates shoot elongation of the shoot primordia. This medium may or may not contain a selection compound.

After every 2 to 3 weeks, the explants are transferred to fresh SEM medium (preferably containing selection) after carefully removing dead tissue. The explants should hold together and not fragment into pieces and retain somewhat healthy. The explants are continued to be transferred until the explant dies or shoots elongate. Elongated shoots >3 cm are removed and placed into RM medium for about 1 week (Methods A and B), or about 2 to 4 weeks depending on the cultivar (Method C) at which time roots began to form. In the case of explants with roots, they are transferred directly into soil. Rooted shoots are transferred to soil and hardened in a growth chamber for 2 to 3 weeks before transferring to the greenhouse. Regenerated plants obtained using this method are fertile and produced on average 500 seeds per plant.

After 5 days of co-cultivation with Agrobacterium tumefaciens transient expression of the gene of interest (GOI) is widespread on the seedling axillary meristem explants especially in the regions wounding during explant preparation (Method A). Explants are placed into shoot induction medium without selection to see how the primary-node responds to shoot induction and regeneration. Thus far, greater than 70% of the explants were formed new shoots at this region. Expression of the GOI is stable after 14 days on SIM, implying integration of the T-DNA into the soybean genome. In addition, preliminary experiments results in the formation of cDNA expressing shoots forming after 3 weeks on SIM.

For Method C, the average regeneration time of a soybean plantlet using the propagated axillary meristem protocol is 14 weeks from explant inoculation. Therefore, this method has a quick regeneration time that leads to fertile, healthy soybean plants.

### Example 9: Pathogen Assay for Soybean

### 9.1. Growth of Plants

12 T1 soybean plants per event and respective controls are potted and grown for 3-4 weeks in the phytochamber (16 h day-und 8 h-night-Rhythm at a temperature of 16° and 22° C. und a humidity of 75%) till the first 2 trifoliate leaves were fully expanded.

### 9.2 Rating for Plant Health

A general rating of plant health is performed before (and partially after) the infection experiment. Only those plants are selected for inoculation that show, in general, a healthy phenotype. Healthy phenotype means normal growth habit, green, fully expanded green leaves, having no or only minor lesions, no obvious yellowing, leaf drop or other stress-associated phenotypes.

| | | | first | |
|---|---|---|---|---|
| | Germination | growth | trifoliate | |
| | rate | phenotype | leaf | older leaves |
| Sideretin | like WT | like WT | like WT | Unclear |
| accumulation: | | | | phenotype: In |
| S8H and | | | | some |
| CYP82C4 and | | | | environments |
| F6H1 and | | | | drying of 2nd |
| CoSy | | | | trifoliate leaves, |
| | | | | but in other |
| | | | | environments no |
| | | | | phenotype visible |
| Scopoletin | Slightly | Smaller | some | Yellowing of |
| accumulation | reduced | plants | yellowing | older leaves |
| (e.g.: F6H1 | | | | |
| and CoSy)(high | | | | |
| accumulating | | | | |
| events) | | | | |

### 9.3 Inoculation

The plants are inoculated with spores of P. pachyrhizi. In order to obtain appropriate spore material for the inoculation, soybean leaves, which are infected with rust 15-20 days ago, are taken 2-3 days before the inoculation and transferred to agar plates (1% agar in H2O). The leaves are placed with their upper side onto the agar, which allowed the fungus to grow through the tissue and to produce very young spores. For the inoculation solution, the spores are knocked off the leaves and are added to a Tween-H2O solution. The counting of spores is performed under a light microscope by means of a Thoma counting chamber. For the inoculation of the plants, the spore suspension is added into a compressed-air operated spray flask and applied uniformly onto the plants or the leaves until the leaf surface is well moisturized. For macroscopic assays a spore density of 1-5×105 spores/ml is used. For the microscopy, a density of >5×105 spores/ml is used. The inoculated plants are placed for 24 hours in a greenhouse chamber with an average of 22° C. and >90% of air humidity. The following cultivation is performed in a chamber with an average of 25° C. and 70% of air humidity.

### Example 10: Microscopical Screening

For the evaluation of the pathogen development, the inoculated leaves of plants are stained with aniline blue 48 hours after infection.

The aniline blue staining serves for the detection of fluorescent substances. During the defense reactions in host interactions and non-host interactions, substances such as phenols, callose or lignin accumulate or are produced and are incorporated at the cell wall either locally in papillae or in the whole cell (hypersensitive reaction, HR). Complexes are formed in association with aniline blue, which lead e.g. in the case of callose to yellow fluorescence. The leaf material is transferred to falcon tubes or dishes containing destaining solution II (ethanol/acetic acid 6/1) and is incubated in a water bath at 90° C. for 10-15 minutes. The destaining solution II is removed immediately thereafter, and the leaves are washed 2× with water. For the staining, the leaves are incubated for 1.5-2 hours in staining solution II (0.05% aniline blue=methyl blue, 0.067 M di-potassium hydrogen phosphate) and analyzed by microscopy immediately thereafter.

The different interaction types are evaluated (counted) by microscopy. An Olympus UV microscope BX61 (incident light) and a UV Longpath filter (excitation: 375/15, Beam splitter: 405 LP) are used. After aniline blue staining, the spores appeared blue under UV light. The papillae can be recognized beneath the fungal appressorium by a green/yellow staining. The hypersensitive reaction (HR) is characterized by a whole cell fluorescence

### Example 11: Evaluating the Susceptibility to Soybean Rust

The progression of the soybean rust disease is scored in percent by the estimation of the diseased area (area which was covered by sporulating uredinia) on the backside (abaxial side) of the leaf. Additionally, the yellowing of the leaf is taken into account. A scheme illustrating the disease rating can be found in WO2016124515 and WO2020120753.

At all 11-12 T1 soybean plants per event and 5 independent events per construct were inoculated with spores of Phakopsora pachyrhizi. The macroscopic disease symptoms of soybean against P. pachyrhizi of the inoculated soybean plants were scored 14 days after inoculation.

The average of the percentage of the leaf area showing fungal colonies or strong yellowing/browning on all leaves was considered as diseased leaf area. At all 59 T1 soybean plants (11-12 per event and 5 independent events per construct) were evaluated in parallel to 18 non-transgenic wild type control plants. Non-transgenic soybean plants grown in parallel to the transgenic plants are used as controls.

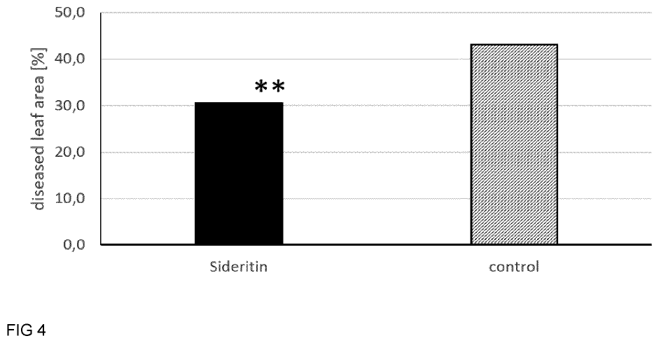

The expression of the construct described in example 7 leads to enhanced resistance of soybean against Phakopsora pachyrhizi by production of sideretin. As shown in FIG. 4 the accumulation of sideretin by expression of S8H and CYP82C4 (and F6H1 and CoSy) leads to an increase of soybean rust resistance by 41%. This increase is statistically significant (p=0,009) as determined by using Students t-test and Levene-Test.

1. Plant cell capable of expressing a heterologous S8H and/or optionally a heterologous CYP82C4 enzyme.

2. Plant cell according to claim 1, wherein, in InterPro nomenclature,
a) the S8H enzyme
is a member of IPR027443 superfamily, and/or
comprises, in N- to C-terminal direction, (i) an IPR026992 non-haem dioxygenase N-terminal domain and (ii) an IPR005123 oxoglutarate/iron-dependent dioxygenase domain and/or an IPR044861 isopenicillin N synthase-like Fe (2+) 2OG dioxygenase domain,
b) the CYP82C4 enzyme
is a member of IPR0363696 superfamily, and/or
comprises an IPR001128 cytochrome P560 domain.

3. Plant cell according to claim 1, wherein
a) the S8H enzyme protein sequence
has 30-90% identity to SEQ ID NO. 1, and only differs from SEQ ID NO. 1 by one, more or all of the following mutations, in the numbering according to SEQ ID NO. 2: A2G, P3I, S4N, D6E, G8Q, N9T, S10T, D18E, V26I, K32S, Q35R, Y37F, I38V, P41L, K42S, D46P, K47T, K48Q, N49K, S51L, K52T, Q55A, -56T, P57Q, K63N, D68Q, D70K, V73A, R78E, P94S, S100L, D103S, A104S, N107E, G110A, P112A, K115E, A117S, V118M, R120L, P127K, E138D, L143I, I149V, V152L, A157S, N165Q, E166P, Y174F, K176N, T177S, K180E, R183K, K184N, L185V, L186V, E187N, V188I, G191E, L193V, E196T, D198E, D199E, S200E, I202M, D203N, A204G, I206M, K208T, F215Y, N221S, T236M, V252L, K254N, V258A, I263V, P264H, V271I, R282K, S294N, T295I, K296G, I301V, I306A, K308N, T310S, E311Q, I313V, Q318E, E321K, K322R, R329K, V332L, N343Q, A344P, I356A, N357E, and/or
is obtainable or obtained by selecting, from the genome of a plant of family Brassicaceae, a gene coding for a protein having 30-90% identity to SEQ ID NO. 1, and/or having 69-100% identity to SEQ ID NO. 2, preferably 83-100%,
b) and/or the CYP82C4 enzyme protein sequence
has 34-90% identity to SEQ ID NO. 3, and only differs from SEQ ID NO. 3 by one, more or all of the following mutations, in the numbering according to SEQ ID NO. 4: P3T, 16F, A7S, L10V, S11P, L12I, I13L, F14V, L15F, Y16V, K17F, V18I, L19A, G21F, -23K, -24S, S27P, S29Y, R30V, E31K, E34A, A36S, G51K, D52E, A61K, K65H, F70M, N71S, I72L, R73Q, R77N, R78E, W85F, E90D, I94V, T104M, V106A, Y115F, P125A, L136I, E145Q, V155I, D156T, I159V, R160K, E161D, N164S, V167F, Q168K, -170G, S172T, R173K, L176M, E178D, R181S, L186M, V190M, V191I, A203G, S204G, A205G, T206S, C207V, -208S, -209S, D210E, G212T, -213E, R216M, R217Q, Q219K, S223A, Q224K, V233T, L238F, F240T, W242S, W243F, L244F, A252E, K255Q, A257G, K258S, A262V, G266R, L268I, E270N, R274Q, V276K, S277F, G278S, K280T, A281K, G283N, Q285S, L292M, Q295A, E297Q, Q299K, N302H, F303L, D308N, T326S, G328S, P341K, E349D, L351I, L353I, K357R, E358D, Q360N, D362E, E363D, K367E, H404Y, A407C, V412I, R423K, W425Y, S426M, N427E, S429N, A430E, Q432R, L437I, S439G, H440E, -442K, D443E, V444F, Q450N, I454M, A466S, F468L, L470M, T476G, L480F, A483S, E485D, L486V, A487K, P489V, L490M, Q492M, T497S, S499N, L514I, T515S, L518I, P519K, A520E, K521E, Y523F, A524V, and/or
is obtainable or obtained by selecting, from the genome of a plant of family Brassicaceae, a gene coding for a protein having 34-90% identity to SEQ ID NO. 3, and/or having 70-100% identity to SEQ ID NO. 4.

4. Plant cell according to claim 1, wherein the plant cell is a transgenic plant cell, and/or wherein the gene coding for the S8H and/or the CYP82C4 enzyme is operably linked to a heterologous promoter and/or terminator.

5. Plant cell according to claim 1, comprising a metabolic pathway for the production of one or more coumarins.

6. Plant cell according to claim 1, wherein expression of the gene coding for the S8H and/or the CYP82C4 enzyme and/or of one or more genes, if present, of the metabolic pathway for production of one or more coumarins is/are directed in a way that expression occurs in root, stem and/or leaf cells.

7. Plant or plant part comprising a plant cell according to claim 1.

8. Plant or plant part according to claim 7, wherein the plant or part thereof exhibits, when compared to a corresponding wild type plant or part thereof,
new or increased production and/or accumulation of (a) fraxetin and optionally sideretin and/or of (b) a derivative thereof, and/or
modified, reduced or removed plant health reducing effects of the production of esculetin, scopoletin and/or isoscopoletin, and/or
reduced, delayed or inhibited germination or growth of a phytopathogenic microorganism on a surface of the plant or plant part, and/or
increased resistance against infection by a phytopathogenic microorganism and/or increased resistance against parasitic plants.

9. Plant or plant part according to claim 7, wherein the plant, plant part or plant cell is soybean, beans, pea, clover, kudzu, lucerne, lentils, lupins, vetches, groundnut, rice, wheat, barley, Arabidopsis, lentil, banana, canola, cotton, potato, maize, sugar cane, alfalfa, sugar beet, sunflower, rapeseed, sorghum, rice, cabbage, tomato, peppers, sugar cane and tobacco, and preferably is a crop plant.

10. Plant progeny obtained by breeding a plant according to claim 7, wherein the progeny comprises the heterologous S8H and/or CYP82C4 gene.

11. Non-propagative plant part or material of a plant or plant part according to claim 7.

12. Product of a plant, plant part or plant cell according to claim 1, wherein the product is obtainable or obtained by
i) collecting a material of said plant, plant part or plant cell, and
ii) disrupting the collected material.

13. Method for improving plant health, comprising the step of conferring or increasing the production and/or accumulation of (a) fraxetin and optionally sideretin and/or of (b) a derivative thereof, in a plant cell, plant part or whole plant in comparison to a respective wild type plant, wild type plant part, or wild type plant cell,
wherein the plant cell, plant part or plant,
by increasing the expression and/or activity of an S8H and/or optionally an CYP82C4 enzyme in the plant, plant part or plant cell, wherein the plant, plant part or plant cell comprises a metabolic pathway for the production of esculetin, scopoletin and/or isoscopoletin.

14. Automated plant selection method, comprising the steps of
i) obtaining, for each seed of a plurality of seeds, a sample comprising genetic material of a tissue body representative for said seed,
ii) determining the presence of a S8H and/or CYP82C4 gene according to any of claims 1-6 in the genetic material, and optionally the presence of one or more genes of a metabolic pathway for production of one or more coumarins,
iii) selecting those seed where the determination in step ii) gave a positive result.

15. (canceled)


SEQUENCE LISTING (6 sequences)

SEQ ID NO: 1 - protein, 356 aa, synthetic construct
MAPSFDDGNSLFNFVVRDGNGVKGMVDSGLSKVPQPYIQPPKERIDKKNASKCEQPPIDLSKLDGPDHDEVVKQIVRAAETLGFFQVVNHGVPVELLESLKDAAHNFFGQPPEKKAVYRKEVSPSPLVKYGTSFVPEKEKALEWKDYISMVYTNDAEALQHWPNECREVALEYLKTSMKMVRKLLEVLMGNLGVELDDSKIDALIGKKMVNMNFYPTCPNPELTVGVGRHSDMGTLTVLLQDGIGGLYVKVDKGEWVEIPPIPGALVINVGDTLQILSNGRYKSAEHRVRTTSTKSRVSIPIFTIPKPTEKIGPLPQVVEKDGVARYREFVFQDYMNNFFGNAHDGKKSLDFARIN

SEQ ID NO: 2 - protein, 357 aa, Arabidopsis thaliana
MGINFEDQTTLFNFVVREGNGVKGMIDSGLSSVPRPFVQPLSERIPTQKALTCEATQPIDLSNLDGPQHKEVAKQIVEAAETLGFFQVVNHGVSVELLELLKSSAHEFFAQAPEEKSMYLKEVSPSKLVKYGTSFVPDKEKAIEWKDYVSMLYTNDSEALQHWPQPCREVALEFLNSSMEMVKNVVNILMENVGVTLEEEKMNGLMGTKMVNMNYYPTCPSPELTVGVGRHSDMGMLTVLLQDGIGGLYVKLDNGEWAEIPPVHGALVINIGDTLQILSNGKYKSAEHRVRTTNIGSRVSVPIFTAPNPSQKVGPLPEVVKRDGVARYKEFLFQDYMNNFFGQPHDGKKSLDFARAE

SEQ ID NO: 3 - protein, 517 aa, synthetic construct
MDPSLIALFLSLIFLYKVLLGKKKSKSREAPEPAGAWPIIGHLHLLGGGDQLLYRTLGAMADKYGPAFNIRLGSRRAFVVSSWEVAKECFTINDKALASRPTTVAAKHMGYNYAVFGFAPYSPFWREMRKIATLELLSNRRLEMLKHVRVSEVDMGIRELYNLWVQNGSRPVLVELKRWLEDLTLNVVVRMVAGKRYFGASATCDDGEARRCQKAISQFFHLIGIFVVSDALPFLWWLDLQGHEKAMKKTAKELDAILEGWLEEHRQRRVSGGKAEGDQDFIDVMLSLQEEGQLSNFQYDADTSIKSTCLALILGGSDTTAGTLTWAISLLLNNPEMLKKAQEELDLHVGKERQVDESDIKNLVYLQAIIKETLRLYPAGPLLGPREAMEDCTVAGYHVPAGTRLVVNVWKIQRDPRVWSNPSAFQPERFLTSHADVDVRGQQFELIPFGSGRRSCPGASFALQVLHLTLARLLHAFELATPLDQPVDMTESPGLTIPKATPLEVLLTPRLPAKLYA

SEQ ID NO: 4 - protein, 524 aa, Arabidopsis thaliana
MDTSLFSLFVPILVFVFIALFKKSKKPKYVKAPAPSGAWPIIGHLHLLGGKEQLLYRTLGKMADHYGPAMSLQLGSNEAFVVSSFEVAKDCFTVNDKALASRPMTAAAKHMGYNFAVFGFAPYSAFWREMRKIATIELLSNRRLQMLKHVRVSEITMGVKDLYSLWFKNGGTKPVMVDLKSWLEDMTLNMIVRMVAGKRYFGGGGSVSSEDTEEAMQCKKAIAKFFHLIGIFTVSDAFPTLSFFDLQGHEKEMKQTGSELDVILERWIENHRQQRKFSGTKENDSDFIDVMMSLAEQGKLSHLQYDANTSIKSTCLALILGGSDTSASTLTWAISLLLNNKEMLKKAQDEIDIHVGRDRNVEDSDIENLVYLQAIIKETLRLYPAGPLLGPREAMEDCTVAGYYVPCGTRLIVNVWKIQRDPKVYMEPNEFRPERFITGEAKEFDVRGQNFELMPFGSGRRSCPGSSLAMQVLHLGLARFLHSFDVKTVMDMPVDMSENPGLTIPKATPLEVLISPRIKEELFV

SEQ ID NO: 5 - DNA, 1074 bp, Arabidopsis thaliana
  source 1..1074: mol_type = unassigned DNA
atgggtatcaatttcgaggaccaaaccacattgttcaatttcgtggtcagagaaggtaacggcgtgaaaggaatgatagactcgggcctctctagtgttccacggcccttcgtccagcctctctctgagcgaatcccgacccaaaaagcccttacgtgtgaggctacccagccaatcgatctttccaacctagacggcccacaacacaaggaggtggccaagcagattgtcgaagctgccgagacacttggcttcttccaggtggtgaaccatggtgtttcggtggagctgctggagttgctaaaatcgtcggctcatgagttttttgcacaagctcctgaggaaaaatcgatgtacctgaaggaagtgagtccaagcaagctggttaagtacggaacgagctttgtgccagataaggagaaggcgattgagtggaaggattatgtgagcatgctttacaccaatgacagtgaggctcttcaacactggcctcaaccatgcagggaggtggcacttgagttcctaaattcatcgatggaaatggtgaaaaatgtagttaacattctgatggagaatgttggagtgacactagaagaagagaaaatgaatggtttgatgggaaccaagatggtcaacatgaactactaccctacttgtccgagccccgagctgaccgtcggcgtcggccgtcactcagacatgggaatgttgaccgttttattacaagacggcattggtggtctctacgttaaactagacaacggtgaatgggccgagatccctcctgtccatggagctttggtcatcaacatcggcgacactttgcagattttgagcaatgggaagtacaaaagtgcggagcatagggttcgaaccacaaacatcggatcaagagtctctgtgcccattttcacggcaccaaatccctcccagaaggtcggtcccttgccggaagttgtgaaacgcgacggagtggctcgttacaaggagtttttgtttcaagactacatgaataacttcttcggccaaccacacgacggcaaaaaatctctcgacttcgcacgtgccgagtga

SEQ ID NO: 6 - DNA, 1575 bp, Arabidopsis thaliana
  source 1..1575: mol_type = unassigned DNA
atggatacttccctcttttctttgtttgttccaatccttgttttcgtttttatcgctcttttcaagaaatcaaagaaaccaaaatatgtaaaagctcctgcaccaagtggtgcatggcccatcatcggccatcttcaccttctcggtggcaaggaacagcttctctaccgaaccttaggaaaaatggctgaccactacggtccagccatgtcgctacaacttgggagcaatgaagcatttgttgtgagcagttttgaggtggctaaagattgttttactgtgaacgacaaggccttggcttcacgtcctatgactgcagctgcaaagcacatgggttacaattttgctgttttcgggtttgccccttatagcgctttctggcgtgagatgcgtaaaatcgcaaccatcgagcttctttctaaccggcggcttcagatgctcaagcacgttcgtgtttctgagatcacaatgggtgtgaaagatttgtattccttgtggttcaagaatggcggtacaaaaccagtaatggttgatctaaagagctggttagaggacatgactctgaacatgatcgtgagaatggtggcaggaaaacgatactttggaggcggaggctcagtatcgtcggaggatactgaagaggcaatgcaatgcaaaaaggccatcgcaaagttctttcacctcatcggtatattcactgtgtcagatgcttttccgacactaagtttttttgatttgcaaggacatgagaaggagatgaagcaaacgggaagcgaattagatgtgatccttgaaagatggattgaaaaccatcgacaacaacgcaaattttcaggaacgaaagagaatgattcagacttcatcgacgttatgatgtcgcttgcggaacaaggaaaactctcgcatctccagtatgatgcaaatactagcatcaaatctacctgcctggcactgattcttggaggaagtgacacttcagcatcaacccttacatgggccatttctcttcttctaaacaataaagaaatgttaaagaaagcacaagatgagatcgacatccacgtcggcagagacaggaacgtcgaggattcagacatagaaaatttggtgtatcttcaagcaattatcaaagaaacattgagattgtatccagctggtcctctcttaggccctcgagaggcgatggaagattgcacggtcgctggttactacgttccgtgcggcacaagactcatagtgaacgtatggaaaatccaaagagatccgaaagtttatatggaaccaaacgagttcagaccagagaggttcattacaggagaagcaaaagagtttgatgtgagaggacaaaactttgagctgatgccatttggttcaggaagaagatcatgcccaggctcttcattggccatgcaagtgcttcatttaggtcttgctcgtttccttcattcatttgacgtgaaaactgttatggatatgcctgttgatatgagtgagaaccctggcttaaccattcctaaagccacgcctcttgaggttctgatcagtccacgtattaaggaagaactttttgtgtga